(19) United States
(12) Patent Application Publication
(10) Pub. No.: US 2026/0283949 A1
(43) Pub. Date: Sep. 24, 2026

(54) STABLE COMPOSITION COMPRISING A HIGH PROTEIN CONCENTRATION

(71) Applicant: Sandoz AG, Basel (CH)

(72) Inventors: Ales ZULA, Ljubljana (SI); Mitja ZIDAR, Ljubljana (SI); Jernej PAVSIC, Ljubljana (SI)

(21) Appl. No.: 19/469,137

(22) PCT Filed: Mar. 30, 2023
(86) PCT No.: PCT/EP2023/058394

§ 371 Date: Sep. 25, 2025

(51) Int. Cl. A61K 9/08; A61K 9/00; A61K 47/02; A61K 47/10; A61K 47/12; A61K 47/18; A61K 47/22; A61K 47/26; C07K 16/28

(52) U.S. Cl. CPC A61K 9/08; A61K 9/0019; A61K 47/02; A61K 47/10; A61K 47/12; A61K 47/183; A61K 47/22; A61K 47/26; C07K 16/2866; C07K 2317/21; C07K 2317/76; C07K 2317/94

(57) ABSTRACT

The present invention relates to aqueous high protein concentration compositions, particularly pharmaceutical compositions. The pharmaceutical compositions are useful for delivery of high concentration of the protein active ingredient, such as an antibody, to a patient without high levels of protein aggregation, or other destabilizing factors. The compositions of the invention comprise about 100 mg/mL or more of protein.

### FIELD OF THE INVENTION

The present invention relates to the field of aqueous high protein concentration compositions, particularly pharmaceutical compositions. More particularly, the present invention relates to the field of high antibody concentration pharmaceutical compositions. More particularly, the present invention relates to methods for stabilizing a composition comprising a high protein concentration, particularly a composition comprising a human antibody, more particularly a composition comprising dupilumab.

### SEQUENCE LISTING

This application includes an electronic sequence listing in a filed named “HEX-015.XML”, created on 8 Mar. 2023 and containing 3 kilobytes, which is hereby incorporated by reference in its entirety for all purposes.

### BACKGROUND OF THE INVENTION

A number of antibodies are approved for therapeutic use in humans and other mammals. Dupilumab (DUPIXENT®), for example, is an interleukin-4 receptor alpha antagonist, available in compositions comprising 175 mg/ml and 150 mg/mL concentrations of dupilumab. Dupilumab is a human monoclonal IgG4 antibody that inhibits interleukin-4 (IL-4) and interleukin-13 (IL-13) signaling by specifically binding to the IL-4Rα subunit shared by the IL-4 and IL-13 receptor complexes and is disclosed in U.S. Pat. No. 7,608,693, which patent is incorporated by reference in its entirety. It inhibits IL-4 signaling via the Type I receptor and both IL-4 and IL-13 signaling through the Type II receptor and is approved for 3 indications: atopic dermatitis, asthma, and chronic rhinosinusitis with nasal polyposis. Formulations of Dupixent are disclosed in U.S. Pat. No. 8,945,559, which patent is incorporated by reference in its entirety.

The concentration of therapeutic antibodies in liquid pharmaceutical compositions varies widely depending, for example, on the route of administration. There is often a need for a composition comprising a high concentration of an antibody when small volumes are desired. For example, a composition comprising a high antibody concentration may be desirable for intravitreal injections or subcutaneous administration. However, a composition comprising a high concentration of antibody may have a short shelf life, and the formulated antibodies may lose biological activity caused by chemical and physical instabilities during storage.

Aggregation, deamidation and oxidation are common causes of antibody degradation, resulting in protein molecules that may be unable to perform the desired biological activity and that are potentially unsafe. Aggregation, for example, is a common degradation pathway of biologics with high protein concentrations (protein concentration about 100 mg/ml and greater) and can potentially cause serious adverse effects by enhancing the protein's immunogenicity, thereby reducing the protein's safety and efficacy. Thus, antibody instability in a composition comprising a high protein concentration must be minimized or prevented.

A composition comprising a high protein concentration can also lead to dramatically increased solution viscosities, which in turn can lead to stability, manufacturing, and delivery challenges, such as increased tissue back-pressure and injection pain. Proteins tend to form viscous solutions at high concentration because of their macromolecular nature and potential for intermolecular interactions. Common inorganic salts have been reported by several studies to reduce mAb viscosity, suggesting that electrostatic interactions strongly contribute to protein-protein interactions (PPI) in many antibodies. However, this is not universally observed. The viscosity-reducing effects of amino acids, especially arginine, have also been reported (Whitaker et al. (2017)).

Although compositions comprising a high protein concentration, including a high antibody concentration, such as DUPIXENT® (150 mg/mL dupilumab and 175 mg/mL dupilumab), are known, there remains a need in the art for further compositions comprising a high protein concentration, such as a high antibody concentration, that are sufficiently stable and suitable for administration to patients, particularly a human.

### SUMMARY OF THE INVENTION

The present invention relates to aqueous high protein concentration compositions, particularly pharmaceutical compositions. More particularly, the present invention relates to pharmaceutical compositions comprising a high antibody concentration. More particularly, the present invention relates to methods for stabilizing a composition comprising a high protein concentration, particularly a composition comprising a human antibody, more particularly a composition comprising dupilumab.

In certain embodiments of the present invention, the invention relates to an aqueous composition comprising (i) dupilumab at a concentration of about 100 mg/mL to about 200 mg/mL, preferably about 150 mg/mL+50 mg/mL to about 175 mg/mL+50 mg/ml; (ii) a histidine buffer, a succinic acid buffer, an adipic acid buffer or no separate buffer; (iii) a component selected from the group consisting of a sugar (preferably sucrose or sorbitol), a non-charged amino acid (preferably methionine or proline), and mixtures thereof; (iv) a surfactant and (v) arginine or lysine.

In certain embodiments the dupilumab concentration is about 150 mg/ml to about 175 mg/mL. In certain embodiments the dupilumab concentration is about 150 mg/ml. In certain embodiments the dupilumab concentration is about 175 mg/ml. In certain embodiments the composition has a pH of about 5.9. In certain embodiments the buffer is adipic acid. In certain embodiments the composition comprises no separate buffer. In certain embodiments the buffer is histidine. In certain embodiments the buffer is succinic acid. In certain embodiments the sugar is sucrose. In certain embodiments the sugar is sorbitol. In certain embodiments the amino acid is methionine. In certain embodiments the amino acid is proline. In certain embodiments the composition comprises arginine. In certain embodiments the composition comprises lysine. In certain embodiments the surfactant is polysorbate 80. In certain embodiments the surfactant is PLX188.

Certain embodiments comprise about 10 mM to about 20 mM histidine buffer. Certain embodiments comprise about 10 mM to about 20 mM succinic acid buffer. Certain embodiments comprise about 10 mM to about 20 mM adipic acid buffer. Certain embodiments comprise no separate buffer.

Certain embodiments comprise about 150 mM to about 170 mM sucrose. Certain embodiments comprise about 150 mM to about 170 mM sorbitol. Certain embodiments comprise about 150 mM methionine. Certain embodiments comprise about 150 mM proline. Certain embodiments comprise a mixture comprising about 150 mM sucrose and about 10 mM methionine. Certain embodiments comprise a mixture comprising about 150 mM sucrose and about 10 mM proline. Certain embodiments comprise a mixture comprising about 150 mM proline and about 10 mM methionine. Certain embodiments comprise a mixture comprising about 150 mM methionine and about 10 mM proline. Certain embodiments comprise about 0.2% polysorbate 80. Certain embodiments comprise about 25 mM to about 150 mM arginine (preferably about 25 mM to about 50 mM arginine, preferably about 25 mM arginine or about 50 mM arginine). Certain embodiments comprise about 50 mM to about 150 mM lysine. Certain embodiments comprise about 75 mM lysine

In certain embodiments of the present invention, the composition comprises (i) dupilumab at a concentration of about 175 mg/ml or about 150 mg/ml; (ii) an about 10 mM histidine/HCl buffer or an about 20 mM histidine/HCl buffer; (iii) a component at a concentration selected from the group consisting of absent, about 150 mM sucrose, about 150 mM sorbitol, about 150 mM methionine, about 150 mM proline, a mixture comprising about 150 mM sucrose and about 10 mM methionine, a mixture comprising about 150 mM sucrose and about 10 mM proline, a mixture comprising about 150 mM methionine and about 10 mM proline, and a mixture comprising about 150 mM proline and about 10 mM methionine; (iv) about 0.2% polysorbate 80; and (v) about 25 mM to about 150 mM arginine (preferably about 25 mM arginine or about 50 mM arginine) or about 50 mM to about 100 mM lysine (preferably about 75 mM). In certain preferred embodiments, the (iii) component is about 150 mM sucrose. In certain preferred embodiments, the (iii) component is about 150 mM sorbitol. In certain preferred embodiments, the (iii) component is about 150 mM methionine. In certain preferred embodiments, the (iii) component is about 150 mM proline. In certain preferred embodiments, the (iii) component is a mixture of about 150 mM sucrose and about 10 mM methionine. In certain preferred embodiments, the (iii) component is a mixture of about 150 mM sucrose and about 10 mM proline. In certain preferred embodiments, the (iii) component is a mixture of about 150 mM methionine and about 10 mM proline. In certain preferred embodiments, the (iii) component is about 150 mM proline and about 10 mM methionine. In certain preferred embodiments, the (iii) component is absent and (v) is about 150 mM arginine, preferably 150 mM arginine HCl.

In certain embodiments of the present invention, the composition comprises dupilumab at a concentration of about 175 mg/ml or about 150 mg/ml; an about 10 mM histidine/HCl buffer or an about 20 mM histidine/HCl buffer; about 150 mM arginine HCl, and about 0.2% polysorbate 80. In certain preferred embodiments, the composition comprises 10 mM histidine/HCl buffer.

In certain preferred embodiments of the present invention, the composition comprises dupilumab at a concentration of about 100 mg/ml to about 200 mg/mL, preferably about 150 mg/mL±50 mg/mL to about 175 mg/mL±50 mg/mL; a histidine/HCl buffer (preferably about 10 mM to about 20 mM, preferably about 10 mM); a polysorbate surfactant (preferably 2 mg/mL polysorbate 80); and about 150 mM arginine, preferably 150 mM arginine HCl. In a preferred embodiment, the protein is an antibody. In a preferred embodiment, the protein is dupilumab.

In certain embodiments of the present invention, the composition comprises (i) dupilumab at a concentration of about 100 mg/mL to about 200 mg/mL, preferably about 150 mg/mL±50 mg/mL to about 175 mg/mL±50 mg/ml; (ii) a component selected from the group consisting of a sugar, preferably sucrose or sorbitol, a non-charged amino acid (preferably methionine or proline), and mixtures thereof; (iv) a polysorbate surfactant and (v) arginine or lysine, wherein the composition does not comprise a separate buffer. In certain preferred embodiments, the (ii) component is sucrose, preferably about 150 mM to about 170 mM sucrose. In certain preferred embodiments, the (ii) component is sorbitol, preferably about 150 mM to about 170 mM sorbitol. In certain preferred embodiments, the (ii) component is a non-charged amino acid. In certain preferred embodiments, the (ii) component is methionine, preferably about 150 mM methionine. In certain preferred embodiments, the (ii) component is proline, preferably about 150 mM proline. In certain preferred embodiments, the (ii) component is a mixture of sucrose and methionine, preferably about 150 mM sucrose and about 10 mM methionine. In certain preferred embodiments, the (ii) component is a mixture of sucrose and proline, preferably about 150 mM sucrose and about 10 mM proline. In certain preferred embodiments, the (ii) component is a mixture of methionine and proline, preferably about 150 mM methionine and about 10 mM proline, or about 150 mM proline and about 10 mM methionine. In a preferred embodiment, the (v) component is arginine, preferably 50 mM arginine. In a preferred embodiment, the protein is an antibody.

In certain embodiments of the present invention, the composition comprises (i) dupilumab at a concentration of about 175 mg/ml or about 150 mg/ml; (ii) a component at a concentration selected from the group consisting of about 150 mM to about 170 mM sucrose, about 150 mM to about 170 mM sorbitol, about 150 mM methionine, about 150 mM proline, a mixture comprising about 150 mM sucrose and about 10 mM methionine, a mixture comprising about 150 mM sucrose and about 10 mM proline, a mixture comprising about 150 mM methionine and about 10 mM proline, and a mixture comprising about 150 mM proline and about 10 mM methionine; (iv) about 0.2% polysorbate 80; and (v) about 25 mM to about 50 mM arginine (preferably about 25 mM arginine or about 50 mM arginine) or about 50 mM to about 100 mM lysine (preferably about 75 mM). In certain preferred embodiments, the (ii) component is about 150 mM to about 170 mM sucrose. In certain embodiments, the (ii) component is about 170 mM sucrose. In certain preferred embodiments, the (ii) component is about 150 mM methionine. In certain preferred embodiments, the (ii) component is about 150 mM proline. In certain preferred embodiments, the (ii) component is a mixture of about 150 mM sucrose and about 10 mM methionine. In certain preferred embodiments, the (ii) component is a mixture of about 150 mM sucrose and about 10 mM proline. In certain preferred embodiments, the (ii) component is a mixture of about 150 mM methionine and about 10 mM proline. In certain preferred embodiments, the (ii) component is a mixture of about 150 mM proline and about 10 mM methionine.

In certain embodiments of the present invention, the composition comprises dupilumab at a concentration of about 150 mg/mL to about 175 mg/mL, preferably about 175 mg/ml or about 150 mg/mL; about 170 mM sucrose; about 0.2% polysorbate 80; and about 50 mM arginine (preferably 50 mM arginine HCl), wherein the composition does not comprise a separate buffer. In certain embodiments, the composition comprises 170 mM sucrose.

In certain embodiments of the present invention, the composition consists of dupilumab at a concentration of about 150 mg/mL to about 175 mg/mL, preferably about 175 mg/ml or about 150 mg/ml; about 170 mM sucrose; about 0.2% polysorbate 80; and about 50 mM arginine (preferably 50 mM arginine HCl). In certain embodiments, the sucrose is 170 mM sucrose.

In certain embodiments of the present invention, the composition comprises (i) dupilumab at a concentration of about 100 mg/mL to about 200 mg/mL, preferably about 150 mg/mL±50 mg/mL to about 175 mg/mL±50 mg/ml; (ii) a succinic acid buffer; (iii) a component selected from the group consisting of a sugar, preferably sucrose or sorbitol, a non-charged amino acid (preferably methionine or proline), and mixtures thereof; and (iv) a polysorbate surfactant. In certain preferred embodiments, the (iii) component is sucrose. In certain preferred embodiments, the (iii) component is sorbitol. In certain preferred embodiments, the (iii) component is a non-charged amino acid. In certain preferred embodiments, the (iii) component is methionine. In certain preferred embodiments, the (iii) component is proline. In certain preferred embodiments, the (iii) component is a mixture of sucrose and methionine. In certain preferred embodiments, the (iii) component is a mixture of sucrose and proline. In certain preferred embodiments, the (iii) component is a mixture of methionine and proline.

In certain embodiments of the present invention, the composition comprises (i) dupilumab at a concentration of about 175 mg/ml or about 150 mg/mL; (ii) a succinic acid buffer; (iii) a component at a concentration selected from the group consisting of about 150 mM sucrose, about 150 mM methionine, about 150 mM proline, a mixture comprising about 150 mM sucrose and about 10 mM methionine, a mixture comprising about 150 mM sucrose and about 10 mM proline, a mixture comprising about 150 mM methionine and about 10 mM proline, and a mixture comprising about 150 mM proline and about 10 mM methionine; and (iv) about 0.2% polysorbate 80. In certain preferred embodiments, the (iii) component is about 150 mM sucrose. In certain preferred embodiments, the (iii) component is about 150 mM methionine. In certain preferred embodiments, the (iii) component is about 150 mM proline. In certain preferred embodiments, the (iii) component is a mixture of about 150 mM sucrose and about 10 mM methionine. In certain preferred embodiments, the (iii) component is a mixture of about 150 mM sucrose and about 10 mM proline. In certain preferred embodiments, the (iii) component is a mixture of about 150 mM methionine and about 10 mM proline. In certain preferred embodiments, the (iii) component is a mixture of about 150 mM proline and about 10 mM methionine.

In certain embodiments of the present invention, the composition comprises (i) dupilumab at a concentration of about 100 mg/ml to about 200 mg/mL, preferably about 150 mg/mL±50 mg/mL to about 175 mg/mL±50 mg/mL; (ii) a succinic acid buffer (preferably about 10 mM to about 20 mM); (iii) a component selected from the group consisting of a sugar (preferably sucrose or sorbitol), a non-charged amino acid (preferably methionine or proline), and mixtures thereof; (iv) a polysorbate surfactant; and (v) arginine or lysine. In certain preferred embodiments, the (iii) component is sucrose. In certain preferred embodiments, the (iii) component is a non-charged amino acid. In certain preferred embodiments, the (iii) component is methionine. In certain preferred embodiments, the (iii) component is proline. In certain preferred embodiments, the (iii) component is a mixture of sucrose and methionine. In certain preferred embodiments, the (iii) component is a mixture of sucrose and proline. In certain preferred embodiments, the (iii) component is a mixture of methionine and proline.

In certain embodiments of the present invention, the composition comprises (i) dupilumab at a concentration of about 175 mg/ml or about 150 mg/ml; (ii) about 10 mM succinic acid/NaOH buffer or about 20 mM succinic acid/NaOH buffer; (iii) a component at a concentration selected from the group consisting of about 150 mM sucrose, about 150 mM sorbitol, about 150 mM methionine, about 150 mM proline, a mixture comprising about 150 mM sucrose and about 10 mM methionine, a mixture comprising about 150 mM sucrose and about 10 mM proline, a mixture comprising about 150 mM methionine and about 10 mM proline, and a mixture comprising about 150 mM proline and about 10 mM methionine; (iv) about 0.2% polysorbate 80; and (v) about 25 mM to about 50 mM arginine (preferably about 25 mM arginine or about 50 mM arginine) or about 50 mM to about 100 mM lysine (preferably about 75 mM lysine). In certain preferred embodiments, the (iii) component is about 150 mM sucrose. In certain preferred embodiments, the (iii) component is about 150 mM methionine. In certain preferred embodiments, the (iii) component is about 150 mM proline. In certain preferred embodiments, the (iii) component is a mixture of about 150 mM sucrose and about 10 mM methionine. In certain preferred embodiments, the (iii) component is a mixture of about 150 mM sucrose and about 10 mM proline. In certain preferred embodiments, the (iii) component is a mixture of about 150 mM methionine and about 10 mM proline. In certain preferred embodiments, the (iii) component is a mixture of about 150 mM proline and about 10 mM methionine. In a preferred embodiment, the protein is an antibody. In a preferred embodiment, the protein is dupilumab.

In certain embodiments of the present invention, the composition comprises (i) dupilumab at a concentration of about 150 mg/mL to about 175 mg/ml (preferably about 150 mg/ml or about 175 mg/mL); (ii) about 10 mM succinic acid/NaOH buffer; (iii) about 150 mM proline; (iv) about 0.2% polysorbate 80; and (v) about 50 mM arginine (preferably 50 mM arginine HCl). In certain preferred embodiments, the (iii) component is 150 mM proline. In a preferred embodiment, the dupilumab is at a concentration of about 150 mg/ml. In a preferred embodiment, the dupilumab is at a concentration of about 175 mg/ml. In certain preferred embodiments, the composition comprises of (i) dupilumab at a concentration of about 150 mg/ml or about 175 mg/ml; (ii) 10 mM succinic acid/NaOH buffer; (iii) 150 mM proline; (iv) 0.2% polysorbate 80; and (v) 50 mM arginine (preferably 50 mM arginine HCl).

In certain embodiments of the present invention, the composition consists of (i) dupilumab at a concentration of about 150 mg/mL to about 175 mg/mL (preferably about 150 mg/ml or about 175 mg/mL); (ii) about 10 mM succinic acid/NaOH buffer; (iii) about 150 mM proline; (iv) about 0.2% polysorbate 80; and (v) about 50 mM arginine (preferably 50 mM arginine HCl). In certain preferred embodiments, the (iii) component is 150 mM proline. In a preferred embodiment, the protein is at a concentration of about 150 mg/ml. In a preferred embodiment, the protein is at a concentration of about 175 mg/ml. In certain preferred embodiments, the composition consists of (i) dupilumab at a concentration of about 150 mg/ml or about 175 mg/ml; (ii) 10 mM succinic acid/NaOH buffer; (iii) 150 mM proline; (iv) 0.2% polysorbate 80; and (v) 50 mM arginine (preferably 50 mM arginine HCl).

In certain embodiments of the present invention, the composition comprises (i) dupilumab at a concentration of about 100 mg/mL to about 200 mg/mL, preferably about 150 mg/mL±50 mg/mL to about 175 mg/mL±50 mg/ml; (ii) an adipic acid buffer (preferably about 10 mM to about 20 mM); (iii) a component selected from the group consisting of a sugar, a non-charged amino acid (preferably methionine or proline), and mixtures thereof; (iv) a polysorbate surfactant; and (v) arginine or lysine. In certain preferred embodiments, the (iii) component is sucrose. In certain preferred embodiments, the (iii) component is sorbitol. In certain preferred embodiments, the (iii) component is a non-charged amino acid. In certain preferred embodiments, the (iii) component is methionine. In certain preferred embodiments, the (iii) component is proline. In certain preferred embodiments, the (iii) component is a mixture of sucrose and methionine. In certain preferred embodiments, the (iii) component is a mixture of sucrose and proline. In certain preferred embodiments, the (iii) component is a mixture of methionine and proline.

In certain embodiments of the present invention, the composition comprises (i) dupilumab at a concentration of about 175 mg/ml or about 150 mg/ml; (ii) about 10 mM adipic acid/NaOH buffer or about 20 mM adipic acid/NaOH buffer; (iii) a component at a concentration selected from the group consisting of about 150 mM sucrose, 150 mM methionine, about 150 mM proline, a mixture comprising about 150 mM sucrose and about 10 mM methionine, a mixture comprising about 150 mM sucrose and about 10 mM proline, a mixture comprising about 150 mM methionine and about 10 mM proline, and a mixture comprising about 150 mM proline and about 10 mM methionine; (iv) about 0.2% polysorbate 80; and (v) about 25 mM to about 50 mM arginine (preferably about 25 mM arginine or about 50 mM arginine) or about 50 mM to about 100 mM lysine (preferably about 75 mM lysine). In certain preferred embodiments, the (iii) component is about 150 mM sucrose. In certain preferred embodiments, the (iii) component is about 150 mM methionine. In certain preferred embodiments, the (iii) component is about 150 mM proline. In certain preferred embodiments, the (iii) component is a mixture of about 150 mM sucrose and about 10 mM methionine. In certain preferred embodiments, the (iii) component is a mixture of about 150 mM sucrose and about 10 mM proline. In certain preferred embodiments, the (iii) component is a mixture of about 150 mM methionine and about 10 mM proline. In certain preferred embodiments, the (iii) component is a mixture of about 150 mM proline and about 10 mM methionine.

In certain particularly preferred embodiments of the present invention, the aqueous high protein concentration composition is a pharmaceutical composition.

In certain embodiments of the present invention, the composition comprises (i) dupilumab at a concentration of 150 or 175 mg/ml; (ii) 10 mM histidine; (iii) 150 mM sucrose; (iv) 2 mg/ml polysorbate 80; and (v) 50 mM arginine.

In certain embodiments of the present invention, the composition comprises (i) dupilumab at a concentration of 150 or 175 mg/ml; (ii) 10 mM histidine; (iii) 150 mM sucrose; (iv) 2 mg/ml polysorbate 80; (v) 50 mM arginine and (vi) 10 mM methionine.

In certain embodiments of the present invention, the composition comprises (i) dupilumab at a concentration of 150 or 175 mg/mL; (ii) 10 mM histidine; (iii) 150 mM sucrose; (iv) 2 mg/ml polysorbate 80; and (v) 75 mM lysine.

In certain embodiments of the present invention, the composition comprises (i) dupilumab at a concentration of 150 or 175 mg/ml; (ii) 10 mM histidine; (iii) 150 mM sorbitol; (iv) 2 mg/ml polysorbate 80; and (v) 50 mM arginine.

In certain embodiments of the present invention, the composition comprises (i) dupilumab at a concentration of 150 or 175 mg/ml; (ii) 10 mM histidine; (iii) 50 mM sucrose; (iv) 2 mg/ml polysorbate 80; and (v) 150 mM lysine.

In certain embodiments of the present invention, the composition comprises (i) dupilumab at a concentration of 150 or 175 mg/ml; (ii) 10 mM histidine; (iii) 50 mM NaCl; (iv) 2 mg/ml polysorbate 80; and (v) 150 mM proline.

In certain embodiments of the present invention, the composition comprises (i) dupilumab at a concentration of 150 or 175 mg/ml; (ii) 10 mM histidine; (iii) 2 mg/ml polysorbate 80; and (iv) 150 mM proline.

In certain embodiments of the present invention, the composition comprises (i) dupilumab at a concentration of 150 or 175 mg/ml; (ii) 20 mM succinic acid and arginine; (iii) 2 mg/ml polysorbate 80; and (iv) 150 mM sucrose.

In certain embodiments of the present invention, the composition comprises (i) dupilumab at a concentration of 150 or 175 mg/ml; (ii) 10 mM succinic acid; (iii) 2 mg/ml polysorbate 80; (iv) 75 mM lysine and (v) 150 mM proline.

In certain embodiments of the present invention, the composition comprises (i) dupilumab at a concentration of 150 or 175 mg/ml; (ii) 10 mM adipic acid; (iii) 150 mM sucrose; (iv) 2 mg/ml polysorbate 80; and (v) 50 mM arginine.

In certain embodiments of the present invention, the composition comprises (i) dupilumab at a concentration of 150 or 175 mg/ml; (ii) 10 mM succinic acid; (iii) 150 mM proline; (iv) 2 mg/ml polysorbate 80; and (v) 50 mM arginine.

In certain embodiments of the present invention, the composition comprises (i) dupilumab at a concentration of 150 or 175 mg/ml; (ii) 170 mM sucrose; (iv) 2 mg/ml polysorbate 80; and (v) 50 mM arginine.

In certain embodiments of the present invention, the composition comprises (i) dupilumab at a concentration of 150 or 175 mg/ml; (ii) 10 mM histidine; (iii) 2 mg/ml polysorbate 80; and (iv) 150 mM arginine.

In certain embodiments of the present invention, the composition comprises (i) dupilumab at a concentration of 150 or 175 mg/mL; (ii) 170 mM sucrose; (iv) 0.5 mg/ml polysorbate 80; and (v) 50 mM arginine.

In certain embodiments of the present invention, the composition comprises (i) dupilumab at a concentration of 150 or 175 mg/ml; (ii) 170 mM sucrose; (iv) 0.5 mg/ml PLX188; and (v) 50 mM arginine.

In certain embodiments of the present invention, the composition comprises (i) dupilumab at a concentration of 150 or 175 mg/ml; (ii) 10 mM histidine; (iii) 2 mg/ml polysorbate 80; and (iv) 150 mM arginine.

The foregoing merely summarizes certain embodiments of the invention and is not intended to be limiting in nature. These and other embodiments of the invention will become apparent from the following description of other embodiments. The detailed description is merely illustrative of the invention and does not limit the scope of the invention, which is defined by the appended claims and equivalents thereof. As would be obvious to one skilled in the art, many variations and modifications of the invention may be effected without departing from the spirit and scope of the novel concepts of the disclosure.

### BRIEF DESCRIPTION OF THE DRAWING

FIG. 1 shows the Arrhenius predictions based on data for dupilumab composition D2 (predicted attribute change, 3 years, 5° C.). R²: HMWs (0.98); LMWs (0.99); Acidic (1.00).

FIG. 2A shows the overall excipient ranking from tested dupilumab compositions D1-D13. Included in the score: high molecular weight (HMW) variants, low molecular weight (LMW) variants and acidic variants.

FIG. 2B shows the main (aggregation/fragmentation) contributions of excipients from tested dupilumab compositions D1-D13. Included in the score: high molecular weight (HMW) variants, low molecular weight (LMW) variants and acidic variants.

### DETAILED DESCRIPTION

The following description of embodiments of the invention is for illustrative purposes only and is not intended to limit the scope of the invention. The patent and scientific literature referred to herein establishes knowledge that is available to those with skill in the art. In the case of inconsistencies, the present disclosure will prevail.

In certain embodiments of the present invention, the composition comprising a high protein concentration is an aqueous pharmaceutical composition.

The high protein concentration compositions of the present invention include aqueous pharmaceutical compositions comprising a high protein concentration. In certain embodiments of the present invention, the composition comprises at least one protein.

In certain embodiments of the present invention, the composition comprises one protein. In certain embodiments, the protein is a polypeptide. In certain embodiments, the protein is an antibody. In certain embodiments, the protein is a human antibody. In a preferred embodiment, the antibody is a monoclonal antibody, preferably a human monoclonal antibody. In a preferred embodiment, the protein is dupilumab.

The term “high protein concentration” is intended to mean a protein concentration greater than about 100 mg/ml±10 mg/mL. In certain embodiments the concentration of the protein is about 100 mg/mL to about 200 mg/ml; about 110 mg/ml to about 190 mg/ml; about 120 mg/mL to about 180 mg/mL; about 150 mg/mL to about 175 mg/mL, about 130 mg/mL to about 170 mg/ml; about 140 mg/mL to about 160 mg/ml; or about 150 mg/ml of a protein. In certain embodiments, the concentration of the protein is about 150 mg/ml to about 175 mg/mL. In certain embodiments, the concentration of the protein is about 90 mg/ml; 95 mg/ml; 100 mg/ml; 105 mg/ml; 110 mg/ml; 115 mg/ml; 120 mg/ml; 125 mg/ml; 130 mg/mL; 135 mg/ml; 140 mg/ml; 145 mg/ml; 150 mg/ml; 155 mg/ml; 160 mg/ml; 165 mg/mL; 170 mg/mL; 175 mg/ml; 180 mg/ml; 185 mg/mL; 190 mg/ml; 195 mg/mL; or about 200 mg/mL. In certain embodiments, the foregoing concentrations include ±10% of each individual indicated concentration (for example, about 100 mg/mL±10 mg/mL to about 200 mg/mL±20 mg/mL). In certain embodiments, the protein concentration is about 150 mg/mL±50 mg/mL to about 175 mg/mL±50 mg/mL. In certain embodiments, the protein concentration is about 175 mg/mL±50 mg/ml. In certain embodiments, the protein concentration is about 175 mg/mL±25 mg/mL, preferably about 175 mg/mL±15 mg/mL. In certain embodiments, the protein concentration is about 150 mg/mL±50 mg/mL. In other certain embodiments, the protein concentration is about 150 mg/mL±25 mg/mL, preferably about 150 mg/mL±15 mg/mL. In certain embodiments, the protein concentration is about 100 mg/mL-150 mg/ml. In certain embodiments, the protein concentration is about 150 mg/mL-200 mg/mL. In certain embodiments, the protein concentration is about 175 mg/ml. In certain embodiments, the protein concentration is about 150 mg/mL. In certain embodiments, the protein concentration is 175 mg/mL. In certain embodiments, the protein concentration is 150 mg/mL. In certain embodiments, the protein concentration is about 100 mg/mL. In certain embodiments, the protein concentration is 100 mg/mL.

As used herein, a pharmaceutical composition is a composition suitable for pharmaceutical use. A composition suitable for pharmaceutical use may be sterile, homogeneous and/or isotonic. Aqueous pharmaceutical compositions may be prepared either directly in an aqueous form, for example in a pre-filled syringe ready for use, or as lyophilisate to be reconstituted shortly before use. As used herein, the term “aqueous pharmaceutical composition” refers to a liquid composition or reconstituted lyophilized composition. In certain embodiments, the aqueous pharmaceutical compositions of the invention are suitable for administration to a human subject. Administration can be by any appropriate route. For example, administration to a patient can be by parenteral routes such as injection (e.g., subcutaneous, intravenous, intramuscular, intraperitoneal, etc.), or percutaneous, mucosal, nasal, pulmonary or oral administration. In certain embodiments, the aqueous pharmaceutical compositions of the invention are suitable for administration by injection. In a specific embodiment, the aqueous pharmaceutical compositions of the invention are suitable for administration by subcutaneous injection.

The term “antibody” as used herein includes whole antibodies and any antigen binding fragment (i.e., “antigen-binding portion”, “antigen binding polypeptide”, or “immunobinder”) or single chain thereof. An “antibody” includes a glycoprotein comprising at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen-binding fragment” of an antibody (or simply “antibody portion”) refers to one or more fragments of an antibody that retain the ability to specifically bind to an antigen. It known in the art that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (ii) a Fd fragment consisting of the VH and CH1 domains; (iv) a Fv fragment consisting of the VL and VH domains of a single arm of an antibody, (v) a single domain or dAb fragment (Ward et al., (1989) Nature 341:544-546), which consists of a VH domain; and (vi) an isolated complementarity determining regions (CDR) or (vii) a combination of two or more isolated CDRs which may optionally be joined by a synthetic linker. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird eta I. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments area screened for utility in the same manner as area intact antibodies. Antigen-binding portions can be produced by recombinant DNA techniques, or by enzymatic or chemical cleavage of intact immunoglobulins. Antibodies can be of different isotype, for example, an IgG (e.g., an IgG1, IgG2, IgG3, or IgG4 subtype), IgA1, IgA2, IgD, IgE or IgM antibody.

In certain embodiments of the present invention, the antibody is dupilumab. The sequence of dupilumab is disclosed in U.S. Pat. No. 7,608,693, which patent is incorporated by reference in its entirety. The sequence of dupilumab is as follows:

| Heavy chain | |
|---|---|
| EVQLVESGGG LEQPGGSLRL SCAGSGFTFR DYAMTWVRQA PGKGLEWVSS | 50 |
| ISGSGGNTYY ADSVKGRFTI SRDNSKNTLY LQMNSLRAED TAVYYCAKDR | 100 |
| LSITIRPRYY GLDVWGQGTT VTVSSASTKG PSVFPLAPCS RSTSESTAAL | 150 |
| GCLVKDYFPE PVTVSWNSGA LTSGVHTFPA VLQSSGLYSL SSVVTVPSSS | 200 |
| LGTKTYTCNV DHKPSNTKVD KRVESKYGPP CPPCPAPEFL GGPSVFLFPP | 250 |
| KPKDTLMISR TPEVTCVVVD VSQEDPEVQF NWYVDGVEVH NAKTKPREEQ | 300 |
| FNSTYRVVSV LTVLHQDWLN GKEYKCKVSN KGLPSSIEKT ISKAKGQPRE | 350 |
| PQVYTLPPSQ EEMTKNQVSL TCLVKGFYPS DIAVEWESNG QPENNYKTTP | 400 |
| PVLDSDGSFF LYSRLTVDKS RWQEGNVFSC SVMHEALHNH YTQKSLSLSL | 450 |
| G | 451 |
| Light chain | |
| DIVMTQSPLS LPVTPGEPAS ISCRSSQSLL YSIGYNYLDW YLQKSGQSPQ | 50 |
| LLIYLGSNRA SGVPDRFSGS GSGTDFTLKI SRVEAEDVGF YYCMQALQTP | 100 |
| YTFGQGTKLE IKRTVAAPSV FIFPPSDEQL KSGTASVVCL LNNFYPREAK | 150 |
| VQWKVDNALQ SGNSQESVTE QDSKDSTYSL SSTLTLSKAD YEKHKVYACE | 200 |
| VTHQGLSSPV TKSFNRGEC. | 219 |

In certain embodiments of the present invention, the antibody comprises at least one amino acid substitution relative to dupilumab. Examples of such amino acid substitutions are known in the art, e.g., in U.S. Pat. No. 10,435,473, which patent is incorporated by reference in its entirety herein.

The term “isolated antibody” is intended to mean an antibody that is substantially free of other antibodies having different antigenic specificities (e.g., an isolated antibody that specifically binds hIL-4Rα is substantially free of antibodies that specifically bind antigens other than hIL4Rα).

The term “specifically binds”, or the like, is intended to mean that an antibody or antigen-binding fragment thereof form a complex with an antigen that is relatively stable under physiologic conditions. Specific binding can be characterized by a dissociation constant of at least about 1×10⁻⁶ M or greater. Methods for determining whether two molecules specifically bind are well known in the art and include, for example equilibrium dialysis, surface plasmon resonance, and the like. An isolated antibody that specifically binds hIL-4Rα, for example, may, however, have cross-reactivity to other antigens, such as IL-4R molecules from other species. In certain embodiments of the present invention, multispecific antibodies (i.e., bispecific) that bind to a target antigen as well as one or more additional antigens are deemed to “specifically bind” to the target antigen.

The amount of protein contained within a protein composition, such as a pharmaceutical composition, of the present invention may vary depending on, the protein, the specific properties desired of the formulation, as well as the particular circumstances and purposes for which the formulation is intended to be used. In certain embodiments, the composition is an aqueous composition. In a preferred embodiment, the protein is an antibody.

Accordingly, in certain embodiments, the composition comprises a dupilumab concentration greater than about 100 mg/ml±10 mg/mL. In certain embodiments the composition comprises a dupilumab concentration of about 100 mg/ml to about 200 mg/ml; about 110 mg/mL to about 190 mg/ml; about 120 mg/mL to about 180 mg/ml; about 150 mg/mL to about 175 mg/mL, about 130 mg/mL to about 170 mg/mL; about 140 mg/mL to about 160 mg/mL; or about 150 mg/mL. In certain embodiments, the composition comprises a dupilumab concentration of about 150 mg/ml to about 175 mg/mL. In certain embodiments, the composition comprises a dupilumab concentration of about 90 mg/mL; 95 mg/ml; 100 mg/ml; 105 mg/mL; 110 mg/ml; 115 mg/mL; 120 mg/ml; 125 mg/ml; 130 mg/ml; 135 mg/ml; 140 mg/mL; 145 mg/ml; 150 mg/mL; 155 mg/ml; 160 mg/ml; 165 mg/ml; 170 mg/mL; 175 mg/ml; 180 mg/ml; 185 mg/ml; 190 mg/ml; 195 mg/mL; or about 200 mg/mL. In certain embodiments, the foregoing concentrations include ±10% of each individual indicated concentration (for example, about 100 mg/mL±10 mg/mL to about 200 mg/mL±20 mg/mL). In certain embodiments, the composition comprises a dupilumab concentration of about 150 mg/mL±50 mg/mL to about 175 mg/mL±50 mg/mL. In certain embodiments, the composition comprises a dupilumab concentration of about 175 mg/mL±50 mg/mL. In certain embodiments, the composition comprises a dupilumab concentration of about 175 mg/mL±25 mg/mL, preferably about 175 mg/mL±15 mg/ml. In certain embodiments, the composition comprises a dupilumab concentration of about 150 mg/mL±50 mg/mL. In other certain embodiments, the composition comprises a dupilumab concentration of about 150 mg/mL±25 mg/mL, preferably about 150 mg/mL±15 mg/mL. In certain embodiments, the composition comprises a dupilumab concentration of about 100 mg/mL-150 mg/ml. In certain embodiments, the composition comprises a dupilumab concentration of about 150 mg/mL-200 mg/mL. In certain embodiments, the composition comprises a dupilumab concentration of about 175 mg/mL. In certain embodiments, the composition comprises a dupilumab concentration of about 150 mg/mL. In certain embodiments, the composition comprises a dupilumab concentration of 175 mg/ml. In certain embodiments, the composition comprises a dupilumab concentration of 150 mg/ml. In certain embodiments, the composition comprises a dupilumab concentration of about 100 mg/mL. In certain embodiments, the composition comprises a dupilumab concentration of 100 mg/mL.

In certain embodiments of the present invention, the composition comprises (i) dupilumab at a concentration greater than about 100 mg/ml±10 mg/mL; (ii) a buffer selected from the group consisting of adipic acid, no separate buffer, histidine, or succinic acid; (iii) a component selected from the group consisting of a sugar, and a non-charged amino acid, and mixtures thereof; (iv) a surfactant and (v) a component selected from the group consisting of arginine and lysine.

In certain embodiments the dupilumab concentration is about 150 mg/ml to about 175 mg/mL. In certain embodiments the dupilumab concentration is about 150 mg/mL. In certain embodiments the dupilumab concentration is about 175 mg/ml. In certain embodiments the composition has a pH of about 5.9. In certain embodiments the buffer is adipic acid. In certain preferred embodiments, the adipic acid buffer is present at a concentration of about 10 mM. In certain embodiments comprises no separate buffer. In certain embodiments the buffer is histidine. In certain preferred embodiments, the histidine acid buffer is present at a concentration of about 10 mM. In certain embodiments the buffer is succinic acid. In certain preferred embodiments, the succinic acid buffer is present at a concentration of about 10 mM to about 20 mM. In certain embodiments the sugar is sucrose. In certain embodiments the sugar is sorbitol. In certain preferred embodiments, the sucrose is present at a concentration of about 150 mM to about 170 mM. In certain preferred embodiments, the sucrose is present at a concentration of about 150 mM. In certain preferred embodiments, the sorbitol is present at a concentration of about 150 mM. In certain preferred embodiments, the sucrose is present at a concentration of about 170 mM. In certain preferred embodiments, the sorbitol is present at a concentration of about 170 mM. In certain embodiments the amino acid is methionine. In certain embodiments the amino acid is proline. In certain preferred embodiments, the amino acid is proline. In certain preferred embodiments, the proline is present at a concentration of about 150 mM. Certain embodiments comprise arginine. In certain preferred embodiments, the arginine is present at a concentration of about 50 mM to 150 mM. In certain preferred embodiments, the arginine is present at a concentration of about 50 mM. Certain embodiments comprise lysine. In certain preferred embodiments, the lysine is present at a concentration of about 75 mM to 150 mM. In certain preferred embodiments, the lysine is present at a concentration of about 75 mM. In certain embodiments the surfactant is polysorbate 80. In certain preferred embodiments, the polysorbate surfactant is polysorbate 80, preferably present at a concentration of about 0.2% or 2 mg/ml. In certain embodiments the surfactant is PLX188. In certain preferred embodiments, the surfactant is PLX188, preferably present at a concentration of about 0.5 mg/ml.

In current pharmaceutical compositions of high protein concentration, histidine buffer is the buffer of choice (Strickley et al., 2021). DUPIXENT®, for example, is approved in two high concentration formulations (150 mg/mL dupilumab and 175 mg/mL dupilumab), both of which are formulated with a histidine/acetate buffer (L-histidine and sodium acetate). Other antibody compositions are summarized in Strickley et al. (2021). It has now been found, however, that certain alternative buffers, or bufferless, and excipient combinations provide stable pharmaceutical compositions, for example under storage conditions and stress conditions.

In certain embodiments of the present invention, the high protein concentration compositions display colloidal stability under storage conditions and stress conditions.

In certain embodiments of the present invention, the selection of components for a stable composition comprising a high protein concentration comprises determining a difference between the target pH of the composition and the isoelectric point (IEP) of the protein itself. A protein formulated close to its IEP will have a low net surface charge (i.e., large positive and negative patches, leading to increased self-interactions). In such a case, a high ionic strength buffer provides more screening of electrostatic attractions between the protein molecules. If a protein is formulated at a pH value far below the IEP (wherein the protein would possess a high net positive surface charge and little or no negative patches), then a low ionic strength buffer is beneficial, since the electrostatic repulsion should not be screened. In certain embodiments of the present invention, where a low ionic strength buffer is used, the composition additionally comprises proline and/or methionine. Examples of high ionic strength buffers include, but are not limited to, succinic acid buffer and adipic acid buffer. Examples of low ionic strength buffers include, but are not limited to, histidine buffer, acetic acid buffer, or bufferless.

The relevant difference (i.e., “close” and “far”) between a protein's IEP and the target pH of a composition can be protein dependent. In certain embodiments of the present invention, a target pH of the composition is “close” to the protein's IEP if the viscosity of the composition decreases upon the addition of a salt (e.g., NaCl); conversely, a target pH of the composition is “far” from the protein's IEP if the viscosity of the composition increases upon the addition of a salt (e.g., NaCl). In certain embodiments of the present invention, a target pH of the composition is “close” to a protein's IEP if the difference between the two values is 2 or less, 1.5 or less, or 0.5 or less. In certain embodiments, a target pH of the composition is “close” to a protein's IEP if the difference between the two values is 1.5 or less.

In certain embodiments, the amino acid is a neutral amino acid. In certain embodiments, the amino acid is selected from the group consisting of histidine, proline, lysine, arginine, methionine, leucine, alanine, tryptophan, and mixtures thereof. In certain embodiments, the amino acid is selected from the group consisting of histidine, proline, lysine, arginine, methionine and mixtures thereof. In certain embodiments, the amino acid is selected from the group consisting of proline, arginine, methionine and lysine and mixtures thereof. In certain embodiments the amino acid is arginine. In certain preferred embodiments, the amino acid is methionine, proline or mixtures thereof. In other preferred embodiments, the amino acid is proline. In other preferred embodiments, the amino acid is methionine. In other embodiments, the amino acid is a mixture of proline and methionine. In other preferred embodiments, the amino acid is lysine. In certain embodiments of the present invention, the amino acid is an L-amino acid.

Suitable buffers for use with the present invention include, but are not limited to, organic acid salts such as salts of adipic acid, citric acid, ascorbic acid, gluconic acid, carbonic acid, tartaric acid, succinic acid, acetic acid or phthalic acid; Tris, tromethamine hydrochloride, or phosphate buffer. In a preferred embodiment of the present invention, the buffer is an organic acid salt, preferably a salt of succinic acid.

In certain embodiments of the present invention, the composition comprises a buffer system that comprises at least two components. In certain embodiments of the invention, the first component is histidine (such as L-histidine), and the second component is an acetate (such as sodium acetate). In certain embodiments of the present invention, the histidine is at a concentration of about 20 mM and the acetate is at a concentration of about 13 mM.

In certain embodiments of the present invention, the ionizable residues in the protein itself provide buffer capacity to the composition, and thus no separate buffer is present. Accordingly, in certain embodiments, the composition comprises dupilumab at a concentration sufficient to provide buffering capacity for the composition and further comprises one or more of the following: other components; and a surfactant. In certain embodiments, the composition comprises dupilumab at a concentration sufficient to provide buffering capacity for the formulation and further comprises the following: first and second other components; and a surfactant. In certain embodiments, the concentration of protein sufficient to provide buffering capacity is about 100 mg/ml and greater. In certain embodiments the concentration of the protein sufficient to provide buffering capacity is about 100 mg/mL to about 200 mg/ml; about 110 mg/mL to about 190 mg/ml; about 120 mg/mL to about 180 mg/ml; about 130 mg/mL to about 170 mg/mL; about 140 mg/mL to about 160 mg/mL; or about 150 mg/ml of dupilumab, in each case the concentration including ±10% of each individual indicated concentration (for example, about 100 mg/mL±10 mg/mL to about 200 mg/mL±20 mg/mL). In certain embodiments, the concentration is about 90 mg/ml; 95 mg/ml; 100 mg/ml; 105 mg/mL; 110 mg/ml; 115 mg/ml; 120 mg/ml; 125 mg/ml; 130 mg/ml; 135 mg/mL; 140 mg/ml; 145 mg/ml; 150 mg/ml; 155 mg/ml; 160 mg/ml; 165 mg/ml; 170 mg/ml; 175 mg/ml; 180 mg/ml; 185 mg/ml; 190 mg/ml; 195 mg/mL; or about 200 mg/ml of dupilumab. In certain embodiments, the concentration is about 175 mg/mL±50 mg/ml of dupilumab. In certain embodiments, the concentration is about 175 mg/mL±25 mg/mL, alternatively 175 mg/mL±15 mg/mL of dupilumab. In certain embodiments, the concentration is about 150 mg/mL±50 mg/mL of dupilumab. In other certain embodiments, the concentration is about 150 mg/mL±25 mg/mL, alternatively 150 mg/mL±15 mg/ml of dupilumab. In certain embodiments, the concentration is about 100 mg/mL-150 mg/ml of dupilumab. In certain embodiments, the concentration is about 175 mg/ml of dupilumab. In certain embodiments, the concentration is about 150 mg/ml of dupilumab. In certain embodiments, the concentration is 175 mg/ml of dupilumab. In certain embodiments, the concentration is 150 mg/mL of dupilumab. In certain embodiments, the concentration is about 100 mg/ml of dupilumab. In certain embodiments, the concentration is 100 mg/ml of dupilumab. In certain embodiments, the concentration of the protein sufficient to provide buffering capacity is about 200 mg/ml or greater.

In certain embodiments, a composition of the invention has a pH between about 5.0 and about 8.0, preferably between about 5.0 and about 7.0. In certain embodiments of the invention, the pH of the composition is about 6.0, preferably 6.0±0.5. In certain embodiments of the invention, the pH of the composition is about 5.9, preferably 5.9±0.1.

In certain embodiments of the present invention, the viscosity of the composition is less than about 25 cPoise, less than about 20 cPoise, less than about 15 cPoise, less than about 14 cPoise, less than about 13 cPoise, less than about 12 cPoise, less than about 11 cPoise, less than about 10 cPoise, or less than about 9 cPoise. In certain embodiments, the viscosity of the composition is about 10 cPoise.

In certain embodiments of the present invention, a the first other component is at a concentration of about 25 mM to about 200 mM, preferably about 25 mM to about 50 mM. In certain embodiments of the invention, the first other component is at a concentration of about 20 mM to about 30 mM, about 40 mM to about 60 mM, or about 65 mM to about 85 mM, preferably about 25 mM, about 50 mM or about 75 mM. In certain embodiments of the present invention, the first other component is at a concentration of about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 105 mM, about 110 mM, about 115 mM, about 120 mM, about 125 mM, about 130 mM, about 135 mM, about 140 mM, about 145 mM, about 150 mm, about 155 mM, or about 160 mM. In certain embodiments of the present invention, the first other component is at a concentration of about 40 mM to about 60 mM, preferably about 50 mM, preferably 50 mM. In certain embodiments of the present invention, the first other component is at a concentration of about 140 mM to about 160 mM, preferably about 150 mM, preferably 150 mM. In certain embodiments of the present invention, the first other component is at a concentration of about 65 mM to about 85 mM, preferably about 75 mM, preferably 75 mM.

In certain embodiments of the present invention, the first other component is an amino acid, at a concentration of about 25 mM to about 200 mM, preferably about 25 mM to about 50 mM. In certain embodiments of the invention, the first other component is an amino acid, at a concentration of about 20 mM to about 30 mM, about 40 mM to about 60 mM, or about 65 mM to about 85 mM, preferably about 25 mM, about 50 mM or about 75 mM. In certain embodiments of the present invention, the first other component is an amino acid, at a concentration of about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 105 mM, about 110 mM, about 115 mM, about 120 mM, about 125 mM, about 130 mM, about 135 mM, about 140 mM, about 145 mM, about 150 mm, about 155 mM, or about 160 mM. In certain embodiments of the present invention, the first other component is an amino acid at a concentration of about 40 mM to about 60 mM, preferably about 50 mM, preferably 50 mM. In certain embodiments of the present invention, the first other component is an amino acid at a concentration of about 140 mM to about 160 mM, preferably about 150 mM, preferably 150 mM. In certain embodiments of the present invention, the first other component is an amino acid at a concentration of about 65 mM to about 85 mM, preferably about 75 mM, preferably 75 mM.

In certain embodiments of the present invention, the first other component is selected from the group consisting of arginine, lysine, proline and methionine, at a concentration of about 25 mM to about 200 mM, preferably about 25 mM to about 50 mM. In certain embodiments of the invention, the first other component is selected from the group consisting of arginine, lysine, proline and methionine, at a concentration of about 20 mM to about 30 mM, about 40 mM to about 60 mM, or about 65 mM to about 85 mM, preferably about 25 mM, about 50 mM or about 75 mM. In certain embodiments of the present invention, the first other component is selected from the group consisting of arginine, lysine, proline and methionine, at a concentration of about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 105 mM, about 110 mM, about 115 mM, about 120 mM, about 125 mM, about 130 mM, about 135 mM, about 140 mM, about 145 mM, about 150 mm, about 155 mM, or about 160 mM. In certain embodiments of the present invention, the first other component is arginine at a concentration of about 40 mM to about 60 mM, preferably about 50 mM, preferably 50 mM. In certain embodiments of the present invention, the first other component is arginine at a concentration of about 140 mM to about 160 mM, preferably about 150 mM, preferably 150 mM. In certain embodiments of the present invention, the first other component is lysine at a concentration of about 65 mM to about 85 mM, preferably about 75 mM, preferably 75 mM.

In certain embodiments of the present invention, the first other component is a salt selected from the group consisting of arginine hydrochloride, sodium thiocyanate, ammonium thiocyanate, ammonium sulfate, ammonium chloride, calcium chloride, zinc chloride and sodium acetate. In certain embodiments of the invention, the first other component is L-arginine hydrochloride.

The compositions of the present invention, such as aqueous pharmaceutical compositions, may comprise one or more second other components in a type and in an amount that stabilizes the protein in the formulation under conditions of storage and/or stress, such as storage time, light and/or thermal stress

A suitable second other component for use with the invention can act, for example, to increase conformational stability of the protein, reduce and/or prevent protein aggregation. The second other component can be ionic or non-ionic (e.g., sugars). As sugars they include, but are not limited top, monosaccharides, e.g., fructose, maltose, galactose, glucose, D-mannose, sorbose and the like; disaccharides, e.g., lactose, sucrose, trehalose, cellobiose, and the like; polysaccharides, e.g., raffinose, melezitose, maltodextrins, dextrans, starches, and the like. In certain embodiments, the second other component is a polyol, such as an alditol, such as mannitol, xylitol, maltitol, lactitol, xylitol sorbitol (glucitol) and the like. For example, the sugar may be sucrose, trehalose, raffinose, maltose, sorbitol or mannitol. In certain embodiments, the sugar may be a sugar alcohol or an amino sugar. In certain embodiments of the invention, the second other component is sucrose. In certain embodiments of the invention, the second other component is sorbitol.

While amino acids will in general decrease viscosity of a high protein concentration composition due to interactions with the protein surface, they can have different effects on conformational stability. According to certain embodiments, the second other component may be an amino acid. In certain embodiments of the present invention, the amino acid is an L-amino acid. One of skill in the art will realize that in general, the stabilizing effect of an amino acid on a protein can be protein-specific, meaning that different amino acids may have different effects on conformational stability of a protein (i.e., some amino acids will have a stabilizing effect, while others may have a destabilizing effect). As such, the stabilizing effect of an amino acid may need to be experimentally tested. The foregoing notwithstanding, it has surprisingly been found that neutral amino acids have a strong conformational stability effect in high protein concentration compositions. Accordingly, in certain embodiments of the present invention, the second other component is a neutral amino acid or a mixture of different neutral amino acids. In certain embodiments, the amino acid is selected from the group consisting of proline, methionine, lysine, and mixtures thereof. In certain preferred embodiments, the amino acid is proline. In other preferred embodiments, the amino acid is methionine. In other embodiments, the amino acid is a mixture of proline and methionine. In certain preferred embodiments, the amino acid is lysine.

In certain embodiments of the present invention, the second other component comprises a sugar and an amino acid, preferably a non-charged amino acid. In certain embodiments, the second other component comprises sucrose and methionine, or sucrose and proline or sucrose and lysine. In certain embodiments of the invention, the second other component comprises sucrose and methionine.

In certain embodiments of the present invention, the total concentration of second other component in a composition is about 50 mM to about 200 mM, about 100 mM to about 200 mM; about 110 mM to about 190 mM; about 120 mM to about 180 mM; about 130 mM to about 170 mM; about 140 mM to about 160 mM; or about 150 mM, in each case the concentration including ±10% of each individual indicated concentration (for example, about 100 mM±10 mM to about 200 mM±20 mM). In certain embodiments, the total concentration of second other component in a composition is about 90 mM; about 95 mM; about 100 mM; about 105 mM; about 110 mM; about 115 mM; about 120 mM; about 125 mM; about 130 mM; about 135 mM; about 140 mM; about 145 mM; about 150 mM; about 155 mM; about 160 mM; about 165 mM; about 170 mM; about 175 mM; about 180 mM; about 185 mM; about 190 mM; about 195 mM; or about 200 mM. In certain embodiments, the total concentration is second other component is about 150 mM±25 mM, about 150 mM±15 mM, about 150 mM±10 mM. In certain embodiments, the total concentration of second other component is about 150 mM or about 160 mM. In certain embodiments, the second other component is about 150 mM sucrose. In certain embodiments, such as those wherein the composition does not comprise a buffer other than the buffering capacity of the protein itself, the second other component is about 170 mM sucrose. In certain embodiments, the second other component is about 150 mM sucrose+about 10 mM methionine. In certain embodiments, the second other component is about 150 mM methionine. In certain embodiments, the second other component is about 150 mM sucrose+about 10 mM proline. In certain embodiments, the second other component is about 150 mM proline. In certain embodiments, the second other component is about 150 mM methionine and about 10 mM proline. In certain embodiments, the second other component is about 150 mM proline and about 10 mM methionine. In certain embodiments, the second other component is about 150 mM sucrose+about 10 mM proline. In certain embodiments, the second other component is about 75 mM lysine. In certain embodiments, the second other component is about 150 mM methionine and about 75 mM lysine.

The term “surfactant” is intended to refer to organic substances having amphipathic structures, i.e., they are composed of groups of opposing solubility tendencies, typically an oil-soluble hydrocarbon chain and a water-soluble ionic group. Surfactants can be classified, depending on the charge of the surface-active moiety, into anionic, cationic and dispersing agents for various pharmaceutical compositions and preparations of biological materials.

In certain embodiments of the present invention, suitable surfactants for use with the invention include, but are not limited to, non-ionic surfactants, ionic surfactants and zwitterionic surfactants. Typical surfactants for use with the invention include, but are not limited to, sorbitan fatty acid esters (e.g. sorbitan monocaprylate, sorbitan monolaurate, sorbitan monopalmitate), sorbitan trioleate, glycerine fatty acid esters (e.g., glycerine monocaprylate, glycerine monomyristate, glycerine monostearate), polyglycerine fatty acid esters (e.g. decaglyceryl monostearate, decaglyceryl distearate, decaglyceryl monolinoleate), polyoxyethylene sorbitan fatty acid esters (e.g. polyoxyethylene sorbitan monolaurate, polyoxyethylene sorbitan monooleate, polyoxyethylene sorbitan monostearate, polyoxyethylene sorbitan monopalmitate, polyoxyethylene sorbitan trioleate, polyoxyethylene sorbitan tristearate), polyoxyethylene sorbitol fatty acid esters (e.g. polyoxyethylene sorbitol tetrastearate, polyoxyethylene sorbitol tetraoleate), polyoxyethylene glycerine fatty acid esters (e.g. polyoxyethylene glyceryl monostearate), polyethylene glycol fatty acid esters (e.g. polyethylene glycol distearate), polyoxyethylene alkyl ethers (e.g. polyoxyethylene lauryl ether), polyoxyethylene polyoxypropylene alkyl ethers (e.g. polyoxyethylene polyoxypropylene glycol, polyoxyethylene polyoxypropylene propyl ether, polyoxyethylene polyoxypropylene cetyl ether), polyoxyethylene alkylphenyl ethers (e.g. polyoxyethylene nonylphenyl ether), polyoxyethylene hydrogenated castor oils (e.g. polyoxyethylene castor oil, polyoxyethylene hydrogenated castor oil), polyoxyethylene beeswax derivatives (e.g. polyoxyethylene sorbitol beeswax), polyoxyethylene lanolin derivatives (e.g. polyoxyethylene lanolin), and polyoxyethylene fatty acid amides (e.g. polyoxyethylene stearic acid amide); Cio-Cis alkyl sulfates (e.g. sodium cetyl sulfate, sodium lauryl sulfate, sodium oleyl sulfate), polyoxyethylene Cio-Cis alkyl ether sulfate with an average of 2 to 4 moles of ethylene oxide units added (e.g. sodium polyoxyethylene lauryl sulfate), and C1-Cis alkyl sulfosuccinate ester salts (e.g. sodium lauryl sulfosuccinate ester); and natural surfactants such as lecithin, glycerophospholipid, sphingophospholipids (e.g. sphingomyelin), and sucrose esters of C12-is fatty acids. In certain embodiments of the invention, the formulation may include one or more of these surfactants. In certain embodiments of the invention, the surfactant is selected from the group consisting of polyoxyethylene sorbitan fatty acid esters e.g., polysorbate 20, 40, 60 or 80. In a preferred embodiment, the surfactant is polysorbate 80.

In certain embodiments of the present invention, the surfactant is at a concentration of from about 0.2%+0.03% to about 1%+0.15% (w/v). In preferred embodiments of the invention, the surfactant is polysorbate 80, which is at a concentration of about 0.2%+0.03% (w/v). In preferred embodiments of the invention, the surfactant is PLX188, which is at a concentration of about 0.05% (w/v).

In certain embodiments of the present invention, a buffer and excipient combination provide a synergistic effect, with respect to the stability of a composition. A synergistic effect can be identified by a finding that a composition performs better than a linear model prediction.

An amino acid may be present in its D- and/or L-form, but the L-form is typical. The amino acid may be present as any suitable salt, e.g., a hydrochloride salt. For example, in accordance with the embodiments of the present invention, arginine is preferably L-arginine hydrochloride.

Other contemplated excipients, which may be utilized in the aqueous pharmaceutical compositions of the invention include, for example, antimicrobial agents, antioxidants, antistatic agents, lipids such as phospholipids or fatty acids, steroids such as cholesterol, protein excipients such as serum albumin (human serum albumin), recombinant human albumin, gelatin, casein, salt-forming counterions such as sodium and the like. These and additional known pharmaceutical excipients and/or additives suitable for use in the compositions of the invention are known in the art, for example, as listed in “The Handbook of Pharmaceutical Excipients, 4^{th} edition, Rowe et al., Eds., American Pharmaceuticals Association (2003); and Remington: the Science and Practice of Pharmacy, 21^{st} edition, Gennaro, Ed., Lippincott Williams & Wilkins (2005).

The aqueous pharmaceutical compositions of the present invention can be used to treat a variety of diseases or disorders, depending on the particular protein (e.g., antibody) contained within the composition. The aqueous pharmaceutical compositions of the present invention can be administered to a human or non-human patient, preferably a human.

Administration will typically be via a syringe. Thus, the invention provides a delivery device (e.g., a syringe, pen, autoinjector) including a pharmaceutical composition of the invention (e.g., a pre-filled syringe, pre-filled pen, autoinjector using a prefilled syringe). In certain preferred embodiments, the syringe is a 1 ml syringe or a 2.25 ml syringe. In certain preferred embodiments, the syringe is a 1 mL BD® Syringe, or a 2.25 mL BD® Syringe. Patients will receive an effective amount of the protein as the principal active ingredient (i.e., an amount that is sufficient to achieve or at least partially achieve the desired effect). A therapeutically effective dose is sufficient if it can produce even an incremental change in the symptoms or conditions associated with the disease or condition. The therapeutically effective dose does not have to completely cure the disease or completely eliminate symptoms. Preferably, the therapeutically effective dose can at least partially arrest the disease and its complications in a patient already suffering from the disease. Amounts effective for use will depend upon the severity of the disease or disorder being treated and/or the general state of the patient being treated.

The dose amount can be readily determined using known dosage adjustment techniques by a physician having ordinary skill in treatment of the disease or condition. The therapeutically effective amount of a protein or antibody used in an aqueous pharmaceutical composition of the invention is determined by taking into account the desired dose volume(s) and mode(s) of administration, for example. Typically, therapeutically effective compositions for injection (e.g., subcutaneous injection) are administered in a dosage ranging from about 100 mg/ml to about 200 mg/mL. Preferably, a dosage used in a method of the invention is about 150 mg/ml to about 200 mg/ml (i.e., about 140, 150, 160, 170, 180, 190, 200, or 210 mg/mL). In a preferred embodiment, the dosage of an antibody is about 150 mg/mL or about 175 mg/mL.

The present invention also provides compositions (i.e., aqueous pharmaceutical compositions) of the invention for use as medicaments, e.g., for use in delivering an antibody to a patient, or for use in treating or ameliorating one or more diseases or disorders.

The present invention further provides a method for delivering an antibody to a patient, comprising a step of administering to the patient an aqueous pharmaceutical composition of the invention.

The pharmaceutical formulations of the present invention typically exhibit high levels of stability. The term “stable” is intended to mean that the proteins within the compositions of the present invention retain an acceptable degree of chemical structure or biological function after storage under defined conditions. A composition may be stable even though the protein contained therein does not maintain 100% of its chemical structure or biological function after storage for a defined amount of time. Under certain circumstances, maintenance of about 90%, about 95%, about 96%, about 97%, about 98% or about 99% of a protein's structure or function after storage for a defined amount of time may be regards as “stable”.

Stability can be measured by various methods known by those of skill in the art (Ma et al., 2020). For example, the percentage of native antibody that remains in a composition after an amount of time in storage at a known temperature, or the percentage of antibody forming in an aggregate within a composition after an amount of time in storage at a known temperature can be determined by size exclusion chromatography (e.g., size exclusion high performance liquid chromatography, SE-HPLC). Other measures of stability include determining the percentage of an antibody that migrates in a more acidic fraction during ion exchange (“acidic form”) than in the main fraction of antibody (“neutral conformation”), wherein the stability is inversely proportional to the fraction of antibody in the acidic form. Alternatively, stability can be measured by determining the biological activity or binding activity of an antibody to its target.

In certain embodiments, the formulations of the invention exhibit low to undetectable levels of antibody aggregation or degradation, with little to no loss of the biological activities during manufacture, preparation, transportation and storage.

In certain embodiments of the present invention, a composition of the invention is stable for at least 1 week, at least 2 weeks, at least 3 weeks, at least 4 weeks, at least 1 month, at least 2 months, at least 3 months, at least 4 months, at least 5 months, at least 6 months, at least 7 months, at least 8 months, at least 9 months, at least 10 months, at least 11 months, at least 12 months, or more. The given temperature at which the composition may be stored when assessing stability can be any temperature from about −80° C. to about 45° C., preferably about 2° C. to about 8° C., about 5° C., about 25° C., or about 45° C.

In certain embodiments of the present invention, a composition is considered stable if at least 90% of the native form of the antibody can be detected in the composition after storage for a defined amount of time at a given temperature. In certain embodiments, at least about 90%, 91%, 92%, 93%, 94%, 95%, 96%, 97%, 98%, 99% or 100% of the native form of the antibody can be detected in a composition after storage for a defined amount of time at given temperature.

In certain embodiments of the present invention, a composition is considered stable if the percentage of aggregated antibody as determined by, for example, SE-HPLC, is at most 5% after storage for a defined amount of time at a given temperature. In certain embodiments, a composition may be considered stable if at most about 5%, 4%, 3%, 2%, 1%, 0.5%, or 0.1% of the antibody can be detected in an aggregate in the composition after storage for a defined amount of time at a given temperature.

In certain embodiments of the present invention, a composition is considered stable if at most 45% of the antibody is in a more acidic form, as detected by cation exchange high performance liquid chromatograph, after storage for a defined amount of time at a given temperature. In certain embodiments of the present invention, a composition is considered stable if at most 35% of the antibody is in a more acidic form, as detected by cation exchange high performance liquid chromatograph, after storage for a defined amount of time at a given temperature. In certain embodiments, a composition may be considered stable if at most about 45%, 40%, 35%, 30%, 25%, 20%, 15%, 10%, 5%, 4%, 3%, 2%, 1%, 0.5%, or 0.1% of the antibody can be detected in an acidic form in the composition after storage for a defined amount of time at a given temperature.

In certain embodiments of the present invention, a composition is considered stable if the antibody maintains its biological activity or a binding affinity. For example, a composition of the present invention may be considered stable if, after storage at e.g., 5° C., 25° C., 45° C., etc., for a defined amount of time the antibody contained in the composition retains its binding affinity to its appropriate ligand with an affinity that is at least 90%, 95%, 96, %, 97%, 98%, 99%, or more of the binding affinity of the antibody prior to the storage. Binding affinity may be determined by appropriate testing, such as ELISA or plasmon resonance. Biological activity may be determined by an appropriate activity assay, such as contacting a cell expressing the appropriate ligand with the composition. Binding activity to the cell may be measured directly, such as by FACS analysis. In a biological assay, for example, the downstream activity produced by an antibody contained in a composition after storage can be compared to the activity produced by an antibody contained in the composition prior to storage. In certain embodiments, a composition is considered stable if at least about 90%, 91%, 92%, 93%, 94%, 95%, 96%, 97%, 98%, 99% or 100% of the biological activity is maintained after storage.

In certain embodiments, a composition of the invention is stable for at least 12 months at 2-8° C. In certain embodiments, a composition of the invention is stable for at least 24 months at 2-8° C. In certain embodiments, a composition of the invention is stable for at least 36 months at 2-8° C.

It has now been surprisingly discovered that stable compositions comprising a high protein concentration can be obtained by formulating the compositions at least in part based on a comparison between the target pH of the composition and the isoelectric point of the protein. It has also been surprisingly discovered that stable compositions comprising a high protein concentration can be obtained using specific composition formulations.

It has also been surprisingly discovered that alternative formulations of dupilumab as compared to the formulation of Dupixent are more stable, in particular having lower aggregate and particle formation. The Dupixent formulations are disclosed in U.S. Pat. No. 8,945,559, which patent is incorporated by reference in its entirety. The current Dupixent formulations are as follows:

| | | | | |
|---|---|---|---|---|
| 300 mg / 2 mL (150) | L-histidine (6.2 mg) / | sucrose (100 mg) | L-arginine hydrochloride | polysorbate 80 (4 mg) |
| | sodium acetate (2 mg) | | (10.5 mg) | |
| 200 mg / 1.14 mL (175) | L-histidine (3.5 mg) / | sucrose (57 mg) | L-arginine | polysorbate 80 (2.3 mg) |
| | sodium acetate | | hydrochloride (12 mg) | |
| | (1.2 mg) | | | |
| 200 / 300 | Histidine / sodium | Sucrose | arginine hydrochloride | polysorbate 80 (E433) |
| | acetate / glacial acetic | | | |
| | acid (E260) | | | |

The compositions of the present invention are useful for the treatment, prevention or amelioration of diseases or disorders responsive to administration of a high concentration protein composition. In certain embodiments, the protein is an antibody. In certain embodiments, the protein is dupilumab. Non-limiting examples of diseases and disorders that can be treated, prevented or ameliorated by the administration of the compositions of the present invention include moderate-to-severe atopic dermatitis, moderate-to-severe asthma, and chronic rhinosinusitis with nasal polyposis. Other non-limiting examples of diseases and disorders that can be treated, prevented or ameliorated by the administration of the compositions of the present invention would be determined by the protein, or antibody, as the case may be, contained in the composition.

Thus, the present invention includes methods of treating, preventing or ameliorating a disease or disorder requiring administration of a high concentration protein composition, including, but not limited to, moderate-to-severe atopic dermatitis, moderate-to-severe asthma, and chronic rhinosinusitis with nasal polyposis. In certain embodiments, the protein is an antibody. In certain embodiments, the protein is dupilumab.

### EXAMPLES

The following examples are for illustrative purposes only and are not intended to limit the scope of the invention.

### Example 1—Composition Preparation

Formulation stock solutions of specific buffer/excipient combinations were added to prepared protein stock solutions with high protein concentration (for example, 230 mg/mL or 200 mg/mL) in water at target pH value to prepare final drug compositions of at least 150 mg/mL. Prepared compositions were filtered through 0.22 μm sterile filter and filled into glass vials. A series of test dupilumab compositions were evaluated after exposure to different storage conditions, together with two reference dupilumab (DUPIXENT®) compositions (Table 1 and Table 2). Compositions D1 and D2 (Table 1) refer to originator compositions for DUPIXENT® 150 mg/mL and 175 mg/mL, respectively. Viscosities for all compositions was about 10 cP.

TABLE 1
All compositions were pH 5.9 and comprised 2 mg/ml polysorbate 80 as a surfactant.

| | Dupilumab | | | |
|---|---|---|---|---|
| | conc. | | First Other | |
| Composition | (mg/mL) | Buffer | Component | Second Other Component |
| D1 | 150 | 20 mM His/Ace | 25 mM Arg | 150 mM sucrose |
| D2 | 175 | 20 mM His/Ace | 50 mM Arg | 150 mM sucrose |
| D3 | 175 | 10 mM His/HCl | 50 mM Arg | 150 mM sucrose |
| D4 | 175 | 10 mM His/HCl | 50 mM Arg | 150 mM sucrose + 10 mM |
| | | | | methionine |
| D5 | 175 | 10 mM His/HCl | 75 mM Lys | 150 mM sucrose |
| D6 | 175 | 10 mM His/HCl | 50 mM Arg | 150 mM sorbitol |
| D7 | 175 | 10 mM His/HCl | 150 mM Lys | 50 mM sucrose |
| D8 | 175 | 10 mM His/HCl | 50 mM NaCl | 150 mM proline |
| D9 | 175 | 10 mM His/HCl | None | 150 mM proline |
| D10 | 175 | 20 mM Succ/Arg | None | 150 mM sucrose |
| D11 | 175 | 20 mM Succ/NaOH | 25 mM Trp | 150 mM sucrose |
| D12 | 175 | 10 mM Succ/NaOH | 75 mM Lys | 150 mM proline |
| D13 | 175 | 10 mM Adip/NaOH | 50 mM Arg | 150 mM sucrose |

His/Ace: histidine/acetate; Arg: L-arginine hydrochloride; His/HCl: Histidine/HCl; Lys: L-lysine; succ/arg: succinic acid buffer + L-arginine (20 mM succinic acid, 35 mM L-arginine; Succ/NaOH: succinic acid buffer + NaOH; Adip/NaOH: adipic acid buffer + NaOH.
D1, D2—DUPIXENT reference formulations.

TABLE 2
All compositions were pH 5.9.

| | Protein | | Buffer | | | | | | Surfactant |
|---|---|---|---|---|---|---|---|---|---|
| | conc. | | conc. | Second other | conc. | First other | conc | | conc. |
| Name | [mg/mL] | Buffer | [mM] | Component | [mM] | Component | [mM] | Surfactant | [mg/mL] |
| DP1_PPM1* | 150 | His/Ac | 20 | Sucrose | 146 | Arg HCl | 50 | PS80 | 2 |
| (*Reference material-like | | | | | | | | | |
| formulation) | | | | | | | | | |
| DP2_PPM1 | 150 | Succinic | 10 | Proline | 150 | Arg HCl | 50 | PS80 | 2 |
| | | acid/NaOH | | | | | | | |
| DP3_PPM1 | 150 | — | — | Sucrose | 170 | Arg HCl | 50 | PS80 | 2 |
| DP3_PPM2 | 150 | — | — | Sucrose | 170 | Arg HCl | 50 | PS80 | 2 |
| DP4_PPM1 | 150 | His/HCl | 10 | — | — | Arg HCl | 150 | PS80 | 2 |
| DP5_PPM1 | 150 | — | — | Sucrose | 170 | Arg HCl | 50 | PS80 | 0.5 |
| DP6_PPM1 | 150 | — | — | Sucrose | 170 | Arg HCl | 50 | PLX188 | 0.5 |
| DP7_PPM3* | 150 | His/Ac | 20 | Sucrose | 170 | Arg HCl | 50 | PS80 | 2 |
| (*Reference material-like | | | | | | | | | |
| formulation) | | | | | | | | | |
| DP8_PPM3 | 150 | Succinic | 10 | Proline | 150 | Arg HCl | 50 | PS80 | 2 |
| | | acid/NaOH | | | | | | | |
| DP9_PPM3 | 150 | — | — | Sucrose | 170 | Arg HCl | 50 | PS80 | 2 |
| DP9_PPM4 | 150 | — | — | Sucrose | 170 | Arg HCl | 50 | PS80 | 2 |
| DP10_PPM3 | 150 | His/HCl | 10 | — | — | Arg HCl | 150 | PS80 | 2; |

His/Ace: histidine/acetate (20 mM L-histidine/13 mM sodium acetate; Arg: L-arginine hydrochloride; His/HCl: Histidine/HCl; PS80: polysorbate 80.
** PPM1 - 1 mL BD ® glass barrel syringe with Hypak plunger stopper from BD ®; PPM2 - 1 mL Ompi EZ-FILL NEXA syringe with NovaPure plunger stopper from West; PPM3 - 2.25 mL BD ® glass barrel syringe with Hypak plunger stopper from BD ®; PPM4 - 2.25 mL Ompi EZ-FILL NEXA syringe with NovaPure plunger stopper from West.

### Analytical Methods

High throughput composition screening studies were performed. Compositions representing various buffers, and other components were screened. Compositions were subjected to different storage conditions and test methods. The measurements enable calculation of an Arrhenius model for long term stability prediction.

Suitable test conditions and test methods for the screening studies are:
(40±2° C.)/(75±5) % r.h.
   VI (Visual Inspection), MFI (Micro-Flow Imaging), SEC (Size Exclusion Chromatography), ICE (Imaging Capillary Electrophoresis), nr CESDS (Non-Reducing Capillary Electrophoresis-Sodium Dodecyl Sulfate), Clarity, UV content, pH, BLI (Bio-Layer Interferometry)
(25±2° C.)/(60±5) % r.h.
   VI, MFI, SEC, ICE, nr CESDS, PS80 content
(5±3° C.)
   VI, MFI, PS80 content
MS (Mechanical stress) (7 days 150 rpm) followed by accelerated storage conditions at (25±2° C.)/(60±5) % r.h. for 2.5 months
   VI, SEC, nrCESDS, MFI
Ambient light exposure (LE) (14 days at RT) followed by accelerated storage conditions at (25±2° C.)/(60±5) % r.h. for 2.5 months
   VI, MFI, SEC, ICE, nr CESDS, PS80, Clarity, pH
Placebos (PPM3 and PPM4 after Mechanical stress-MFI only)

Raw data from the individual stability conditions were assessed using an in-house Excel-based tool (eTREADS—Tool for Rapid Evaluation of Analytical Data from Stability Studies). For each analytical method, assessment of samples from the same storage condition is based on their analytical results, with method variability taken into account (standard deviation, relative standard deviation or specified method variability).

For each category (e.g. SEC-sum of aggregates, MFI-sum of subvisible particles ≥10 μm/mL) of analytical method (e.g. SEC, MFI), different assessment approaches could be defined, according to the type of stability study (formulation study or stability study), the expected change of quality attribute over time (increase, decrease, both), similarity of initial results for a given quality attribute, or use of method variability (standard deviation (SD), relative standard deviation (RSD) or accepted intervals). Assessment types are described in detail in Table 3. Each result is assigned an SD score (i.e., a real number with one decimal place), which indicates how big a difference is between a particular result and results of other samples when method variability is taken into account. The higher the SD score, the higher the difference is between a particular result of interest and the best result. The SD scores allow for a comprehensive overview for all samples and analytical methods from a defined storage condition and sample time.

TABLE 3
Assessment Approaches and Generation of SD Score for Assessment Type

| | | Expected change | | |
|---|---|---|---|---|
| | | in QA on storage | | |
| | | condition and | | |
| | | similarity of results | | |
| | | at t0 between | | |
| | Most | different | | |
| | appropriate | formulations | | |
| | for specific | (applicable only | Method | |
| | type of | when formulations | variability | |
| Assessment type | study | are compared) | used | How SD score is generated |
| 1- Increase in QA | Formulation | Increase in QA (e.g. | SD | The sample with the lowest measured value of quality attribute (e.g., |
| at pp (with SD) - | study | aggregates, sub- | | 5.0%) receives a SD score of 0. The analytical results of each other |
| compare | | visible particles), | | tested sample (e.g., 9.1%) are directly compared to the sample with |
| formulations | | similar t0 results | | the lowest result. The difference between them is then expressed as |
| | | between | | the highest number of ±SD intervals applied to both results that still |
| | | formulations | | allows the statistically significant difference between results. In the |
| | | | | case where SD = 1.0%, the highest number of ±SD intervals that |
| | | | | still separate those two values is 2.0, since those two results can still |
| | | | | be considered as not statistically different when ±2.1 SD intervals |
| | | | | would be applied to both results but are considered different when ±2.0 |
| | | | | SD intervals are taken into account. Such result (e.g., 9.1%) |
| | | | | would in turn receive a SD score = 2.0, since this is the highest |
| | | | | amount of ±SD intervals that would still result in a statistically |
| | | | | significant difference when compared to the lowest result (5%). |
| 2- Decrease in QA | Formulation | Decrease in QA (e.g. | SD | The sample with the highest measured value of a quality attribute |
| at pp (with SD) - | study | purity of mAb), | | (e.g., 9.1%) receives a SD score of 0. The analytical results of each |
| compare | | similar t0 results | | other tested sample (e.g., 5.0%) are directly compared to that |
| formulations | | between | | sample with the highest result. The difference between them is then |
| | | formulations | | expressed as the highest number of ±SD intervals applied to both |
| | | | | results that still allows the statistically significant difference between |
| | | | | results. In the case where SD = 1.0%, the highest number of ±SD |
| | | | | intervals that still separate those two values is 2.0, since those two |
| | | | | results can still be considered as not statistically different when ±2.1 |
| | | | | SD intervals would be applied to both results but are considered |
| | | | | different when ±2.0 SD intervals are taken into account. Such |
| | | | | result (5.0%) would in turn receive a SD score = 2.0, since this is |
| | | | | the highest amount of ±SD intervals that would still result in a |
| | | | | statistically significant difference when compared to the highest |
| | | | | result (9.1%). |
| 3- Increase in QA | Formulation | Increase in QA (e.g. | RSD | The sample with the lowest measured value of a quality attribute |
| at pp (with RSD) - | study | aggregates, sub- | | (e.g., 10.0%) receives a SD score of 0. The analytical results of |
| compare | | visible particles), | | each other tested sample (e.g., 10.5%) are directly compared to the |
| formulations | | similar t0 results | | sample with lowest result. The difference between them is then |
| | | between | | expressed as the highest number of ±RSD intervals applied to both |
| | | formulations | | results that still allows the statistically significant difference between |
| | | | | the results. In the case where RSD = 1.0%, the highest number of ±SD |
| | | | | intervals that still separate those two values is 2.4, since those |
| | | | | two results can still be considered as not statistically different when ±2.5 |
| | | | | RSD intervals would be applied to both results but are |
| | | | | considered different when ±2.4 RSD intervals are taken into |
| | | | | account. Such result (10.5%) would in turn receive a SD score = |
| | | | | 2.4, since this is the highest amount of ±RSD intervals that would |
| | | | | still result in a statistically significant difference when compared to |
| | | | | the lowest result (10.0%). |
| 4- Decrease in QA | Formulation | Decrease in QA (e.g. | RSD | The sample with the highest measured value of a quality attribute |
| at pp (with RSD) - | study | purity of mAb), | | (e.g., 10.0%) receives a SD score of 0. The analytical results of |
| compare | | similar t0 results | | each other tested sample (e.g., 9.5%) are directly compared to the |
| formulations | | between | | sample with highest result. The difference between them is then |
| | | formulations | | expressed as the highest number of ±RSD intervals applied to both |
| | | | | results that still allows the statistically significant difference between |
| | | | | results. In the case where RSD = 1.0%, the highest number of ±SD |
| | | | | intervals that still separate those two values is 2.5, since those |
| | | | | two results can still be considered as not statistically different when ±2.6 |
| | | | | RSD intervals would be applied to both results but are |
| | | | | considered different when ±2.5 RSD intervals are taken into |
| | | | | account. Such result (9.5%) would in turn receive a SD score = 2.5, |
| | | | | since this is the highest amount of ±RSD intervals that would still |
| | | | | result in a statistically significant difference when compared to the |
| | | | | highest result (10.0%). |
| 5- Increase in QA | Formulation | Increase in QA (e.g. | SD | The increase in a quality attribute is first calculated for each sample |
| between pp and t0 | study | aggregates, sub- | | as the difference in results at time zero (t0) and a specific pull point |
| (with SD) - | | visible particles), | | (pp). Samples with a decrease are considered as increase = 0. The |
| compare | | significantly different | | sample with the lowest measured increase in the quality attribute |
| formulations | | t0 results between | | (e.g., 5.0% of increase) receives a SD score of 0. The calculated |
| | | formulations | | increase of each other tested sample (e.g., 12%) is directly |
| | | | | compared to the increase of that sample with the smallest increase. |
| | | | | Since both measurements (t0 and that at pp) have the same |
| | | | | measurement uncertainty that is derived from (SDₘₑₐₛᵤᵣₑₘₑₙₜ), the |
| | | | | uncertainty of increase (SD_{increase}) is therefore calculated as the |
| | | | | square root of (SDₘₑₐₛᵤᵣₑₘₑₙₜ² + SDₘₑₐₛᵤᵣₑₘₑₙₜ²), which turns out |
| | | | | as approximately 1.4142 × SDₘₑₐₛᵤᵣₑₘₑₙₜ. The difference between |
| | | | | both increases is then expressed as the highest number of ±SD_{increase} |
| | | | | intervals applied to both results that still allows the |
| | | | | statistically significant difference between results. In the case where |
| | | | | SDₘₑₐₛᵤᵣₑₘₑₙₜ = 1.0%, the highest number of ±SD_{increase} intervals |
| | | | | that still separate those two values (5% and 12%) is 2.4, since those |
| | | | | two results can still be considered as not statistically different when ±2.5 |
| | | | | SDincrease intervals would be applied to both results but are |
| | | | | considered different when ±2.4 SD_{increase} intervals are taken into |
| | | | | account. Such result (e.g., 12% of increase) would in turn receive a |
| | | | | SD score = 2.4, since this is the highest amount of ±SD_{increase} |
| | | | | intervals that would still result in a statistically significant difference |
| | | | | when compared to the lowest increase (5%). |
| 6- Decrease in QA | Formulation | Decrease in QA, | SD | The decrease in a quality attribute is first calculated for each sample |
| between pp and t0 | study | significantly different | | as the difference in results at time zero (t0) and a specific pull point |
| (with SD) - | | t0 results between | | (pp). Samples with an increase are considered as decrease = 0. |
| compare | | formulations | | The sample with the lowest measured decrease in the quality |
| formulations | | | | attribute (e.g., 5.0% of decrease) receives a SD score of 0. The |
| | | | | calculated decrease of each other tested sample (e.g., 12%) is |
| | | | | directly compared to the decrease of the sample with the smallest |
| | | | | decrease. Since both measurements (t0 and that at pp) have the |
| | | | | same measurement uncertainty that is derived from control chart |
| | | | | (SDₘₑₐₛᵤᵣₑₘₑₙₜ), the uncertainty of decrease (SD_{decrease}) is |
| | | | | therefore calculated as the square root of (SDₘₑₐₛᵤᵣₑₘₑₙₜ² + |
| | | | | SDₘₑₐₛᵤᵣₑₘₑₙₜ²), which turns out as approximately 1.4142 × |
| | | | | SDₘₑₐₛᵤᵣₑₘₑₙₜ. The difference between both decreases is then |
| | | | | expressed as the highest number of ±SD_{decrease} intervals applied to |
| | | | | both results that still allows the statistically significant difference |
| | | | | between results. In the case where SDₘₑₐₛᵤᵣₑₘₑₙₜ = 1.0%, the |
| | | | | highest number of ±SD_{decrease} intervals that still separate those two |
| | | | | values (5% and 12%) is 2.4, since those two results can still be |
| | | | | considered as not statistically different when ±2.5 SD_{decrease} |
| | | | | intervals would be applied to both results but are considered different |
| | | | | when ±2.4 SD_{decrease} intervals are taken into account. Such result |
| | | | | (12% of decrease) would in turn receive a SD score = 2.4, since this |
| | | | | is the highest amount of ±SD_{decrease} intervals that would still result |
| | | | | in a statistically significant difference when compared to the lowest |
| | | | | decrease (5%). |
| 7- Decrease in QA | Stability | Decrease in QA (e.g. | SD | For each sample, the analytical result (e.g., 5.0%) from any pull |
| between pp and t0 | study | purity of mAb) | | point and storage condition (e.g., 3-m pp from 5° C., 6-m pp from 5° |
| (with SD) - | | | | C.) is compared directly with the t0 result (e.g. 9.1%) of the |
| individual | | | | corresponding sample. In the case of an increase, the SD score = 0 |
| comparison | | | | is applied. In the case of a decrease, the difference between these |
| | | | | two results is then expressed as the highest number of ±SD |
| | | | | intervals applied to both results that still allows the statistically |
| | | | | significant difference between results. In the case where SD = 1.0%, |
| | | | | the highest number of ±SD intervals that still separate those two |
| | | | | values is 2.0, since those two results can still be considered as not |
| | | | | statistically different when ±2.1 SD intervals would be applied to |
| | | | | both results but are considered different when ±2.0 SD intervals are |
| | | | | taken into account. Such result (5.0%) would in turn receive a SD |
| | | | | score = 2.0, since this is the highest amount of ±SD intervals that |
| | | | | would still result in a statistically significant difference when |
| | | | | compared to the result at t0 (9.1%). |
| 8- Increase in QA | Stability | Increase in QA (e.g. | SD | For each sample, the analytical result (e.g., 9.1%) from any pull |
| between pp and t0 | study | aggregates, sub- | | point and storage condition (e.g., 3-m pp from 5° C., 6-m pp from 5° |
| (with SD) - | | visible particles) | | C.) is compared directly with the t0 result (e.g., 5.0%) of the |
| individual | | | | corresponding sample. In case the of a decrease, SD score = 0 is |
| comparison | | | | applied. In the case of an increase, the difference between these |
| | | | | two results is then expressed as the highest number of ±SD |
| | | | | intervals applied to both results that still allows the statistically |
| | | | | significant difference between results. In the case where SD = 1.0%, |
| | | | | the highest number of ±SD intervals that still separate those two |
| | | | | values is 2.0, since those two results can still be considered as not |
| | | | | statistically different when ±2.1 SD intervals would be applied to |
| | | | | both results but are considered different when ±2.0 SD intervals are |
| | | | | taken into account. Such a result (9.1%) would in turn receive a SD |
| | | | | score = 2.0, since this is the highest amount of ±SD intervals that |
| | | | | would still result in a statistically significant difference when |
| | | | | compared to the result at t0 (5.0%). |
| 9- Decrease in QA | Stability | Decrease in QA (e.g. | RSD | For each sample, the analytical result (e.g., 9.5%) from any pull |
| between pp and t0 | study | purity of mAb) | | point (pp) and storage condition (e.g., 3-m pp from 5° C., 6-m pp from |
| (with RSD) - | | | | 5° C.) is compared directly with the time zero (t0) result (e.g., 10%) |
| individual | | | | of the corresponding sample. In the case of an increase, SD score = |
| comparison | | | | 0 is applied. In the case of a decrease, the difference between these |
| | | | | two results is then expressed as the highest number of ±RSD |
| | | | | intervals applied to both results that still allows the statistically |
| | | | | significant difference between results. In the case where RSD = 1.0%, |
| | | | | the highest number of ±RSD intervals that still separate those |
| | | | | two values is 2.5, since those two results can still be considered as |
| | | | | not statistically different when ±2.6 RSD intervals would be applied |
| | | | | to both results but are considered different when ±2.5 RSD intervals |
| | | | | are taken into account. Such result (9.5%) would in turn receive a |
| | | | | SD score = 2.5, since this is the highest amount of ±RSD intervals |
| | | | | that would still result in a statistically significant difference when |
| | | | | compared to the result at t0 (10.0%). |
| 10- Increase in QA | Stability | Increase in QA (e.g. | RSD | For each sample, the analytical result (e.g., 10.5%) from any pull |
| between pp and t0 | study | aggregates, sub- | | point (pp) and storage condition (e.g., 3-m pp from 5° C., 6-m pp from |
| (with RSD) - | | visible particles) | | 5° C.) is compared directly with the time zero (t0) result (e.g., 10%) |
| individual | | | | of the corresponding sample. In the case of a decrease, SD score = |
| comparison | | | | 0 is applied. In the case of an increase, the difference between these |
| | | | | two results is then expressed as the highest number of ±RSD |
| | | | | intervals applied to both results that still allows the statistically |
| | | | | significant difference between results. In the case where RSD = 1.0%, |
| | | | | the highest number of ±RSD intervals that still separate those |
| | | | | two values is 2.4, since those two results can still be considered as |
| | | | | not statistically different when ±2.5 RSD intervals would be applied |
| | | | | to both results but are considered different when ±2.4 RSD intervals |
| | | | | are taken into account. Such result (10.5%) would in turn receive a |
| | | | | SD score = 2.4, since this is the highest amount of ±RSD intervals |
| | | | | that would still result in a statistically significant difference when |
| | | | | compared to the result at t0 (10.0%). |
| 11- Change in QA | Formulation | Increase or | RSD | For each sample, the analytical result (e.g., 10.5%) from any pull |
| between pp and t0 | study and | decrease in QA (e.g. | | point (pp) and storage condition (e.g., 3-m pp from 5° C., 6-m pp from |
| (with RSD) - | stability study | pH, UV) | | 5° C.) is compared directly with the time zero (t0) result (e.g., 10%) |
| individual | | | | of the corresponding sample. In the case of an increase or a |
| comparison | | | | decrease, the difference between these two results is then |
| | | | | expressed as the highest number of ±RSD intervals applied to both |
| | | | | results that still allows the statistically significant difference between |
| | | | | the results. In the case where RSD = 1.0%, the highest number of ±RSD |
| | | | | intervals that still separate those two values is 2.4, since those |
| | | | | two results can still be considered as not statistically different when ±2.5 |
| | | | | RSD intervals would be applied to both results but are |
| | | | | considered different when ±2.4 RSD intervals are taken into |
| | | | | account. Such result (10.5%) would in turn receive a SD score = |
| | | | | 2.4, since this is the highest amount of ±RSD intervals that would |
| | | | | still result in a statistically significant difference when compared to |
| | | | | the result at t0 (10.0%). |
| 12- Change in QA | Formulation | Increase or | SD | For each sample, the analytical result (e.g., 9.1%) from any pull |
| between pp and t0 | study and | decrease in QA (e.g. | | point (pp) and storage condition (e.g. 3-m pp from 5° C., 6-m pp from |
| (with SD) - | stability study | pH, UV) | | 5° C.) is compared directly with the time zero (t0) result (e.g., 5.0%) |
| individual | | | | of the corresponding sample. In the case of an increase or a |
| comparison | | | | decrease, the difference between these two results is then |
| | | | | expressed as the highest number of ±SD intervals applied to both |
| | | | | results that still allows the statistically significant difference between |
| | | | | the results. In the case where SD = 1.0%, the highest number of ±SD |
| | | | | intervals that still separate those two values is 2.0, since those |
| | | | | two results can still be considered as not statistically different when ±2.1 |
| | | | | SD intervals would be applied to both results but are considered |
| | | | | different when ±2.0 RSD intervals are taken into account. Such |
| | | | | result (9.1%) would in turn receive a SD score = 2.0, since this is the |
| | | | | highest amount of ±SD intervals that would still result in a |
| | | | | statistically significant difference when compared to the result at t0 |
| | | | | (5.0%). |
| 13- Change in QA | Formulation | Increase or | Lower and | Each result within specified limits would receive SD score = 0. Each |
| outside predefined | study and | decrease in QA (e.g. | upper limit | result outside specified limits would receive SD score = 5. |
| range - individual | stability study | bioassay with | | |
| comparison | | defined acceptance | | |
| | | limits) | | |

SD—Standard deviation; RSD—Relative standard deviation.

### Example 2—Size Exclusion Chromatography (SEC) Analysis

Proteins in high protein concentrated solutions are prone to aggregation.

In SEC method proteins are separated on the basis of their difference in hydrodynamic volume in the mobile phase by diffusion in and out of stagnant pores. Main application is the determination of protein aggregates.

The eTreads evaluation of the data for SEC analysis of compositions D1-D13 is presented in Table 4.

### Example 3—Capillary Electrophoresis-SDS (nrCE-SDS) Analysis

Protein is denatured with Sodium dodecyl sulfate (SDS) and alkylated with N-ethylmaleimide. SDS masks the intrinsic charge of the proteins and forms complexes with a constant charge per unit mass. These complexes can be separated according to their size in an electric field by adding a hydrophilic sieving polymer to the separation buffer

Data for nrCE-SDS analysis of compositions D1-D13 are presented in Table 5.

### Example 4—Charge Variant (ICE) Analysis

Imaged capillary isoelectric focusing (icIEF or ICE) is used for determination of charge variants and purity of proteins. icIEF separates protein species (variants) based on their charge differences in a pH gradient. The protein is focused in a capillary column under high voltage. The focusing process is monitored in a real-time mode using whole column imaging detection (WCID) system. The resolved charge variants appear as electrophoretic peaks, which are detected using ultraviolet light absorbance at a fixed wavelength of 280 nm. Charge variants and purity are determined as a percentage of the total peak area obtained for the sample in each electropherogram.

Data for iCE analysis of compositions D1-D13 are presented in Table 6.

### Example 5—Micro-Flow Imaging (MFI) Method

MFI is a flow microscopy technology, where bright field images are captured in successive frames as a continuous sample stream passes through a flow cell centered in the field-of-view of a custom magnification system having a well-characterized and extended depth-of-field. It allows to detect particles in the sub-visible range and report the concentration of particles detected in different size ranges.

### Example 6—Biolayer Interferometry (BLI) Method

Bio-Layer Interferometry (BLI) is a label-free technology for measuring biomolecular interactions. It is an optical analytical technique that analyzes the interference pattern of white light reflected from two surfaces: a layer of immobilized protein on the biosensor tip, and an internal reference layer. Any change in the number of molecules bound to the biosensor tip causes a shift in the interference pattern that can be measured in real-time.

Individual formulations showed no difference in pH or clarity during the stability studies.

TABLE 4
SEC Analysis (40° C., 11 weeks)

Analytical Method
SEC
Type of Assessment

| | Type 5 | Type 5 | Type 6 |
|---|---|---|---|
| | Sum of | Degradation | Main |
| Composition | Aggregates | Products | Peak |
| D1 | 1.1 | 0.2 | 0.4 |
| D2 | 0.0 | 0.2 | 0.0 |
| D3 | 2.4 | 0.1 | 0.8 |
| D4 | 0.3 | 0.0 | 0.0 |
| D5 | 8.7 | 0.1 | 3.0 |
| D6 | 15.7 | 0.4 | 5.8 |
| D7 | 15.3 | 0.4 | 5.7 |
| D8 | 9.9 | 0.3 | 3.6 |
| D9 | 8.4 | 0.0 | 2.8 |
| D10 | 4.5 | 0.0 | 1.4 |
| D11 | 18.3 | 0.2 | 6.5 |
| D12 | 2.9 | 0.0 | 0.9 |
| D13 | 2.4 | 0.0 | 0.7 |

TABLE 5
nrCE-SDS Analysis (40° C., 11 weeks)

Analytical Method
nrCE-SDS
Type of Assessment

| | Type 5 | Type 5 | Type 6 |
|---|---|---|---|
| Composition | Sum of LMW | Sum of HMW | Main Peak |
| D1 | 0.2 | 0.0 | 0.0 |
| D2 | 0.2 | 0.1 | 0.1 |
| D3 | 1.1 | 0.4 | 0.9 |
| D4 | 0.5 | 0.2 | 0.4 |
| D5 | 1.6 | 1.6 | 2.5 |
| D6 | 2.6 | 1.8 | 3.5 |
| D7 | 3.5 | 2.4 | 4.5 |
| D8 | 1.9 | 2.2 | 3.2 |
| D9 | 1.1 | 1.8 | 2.2 |
| D10 | 0.0 | 0.8 | 0.4 |
| D11 | 0.9 | 3.2 | 3.1 |
| D12 | 1.2 | 0.7 | 1.4 |
| D13 | 0.8 | 0.8 | 1.1 |

TABLE 6
iCE Analysis (40° C., 11 weeks)

Analytical Method
iCE
Type of Assessment

| | Type 5 | Type 5 | Type 6 |
|---|---|---|---|
| | Sum of | Sum of | Main |
| Composition | Basic Peaks | Acidic Peaks | Peak |
| D1 | 2.7 | 1.4 | 1.2 |
| D2 | 1.6 | 1.0 | 0.4 |
| D3 | 1.1 | 0.7 | 0.0 |
| D4 | 2.1 | 1.2 | 0.8 |
| D5 | 1.5 | 1.6 | 1.0 |
| D6 | 1.5 | 1.2 | 0.5 |
| D7 | 2.5 | 1.2 | 1.2 |
| D8 | 2.9 | 1.0 | 0.7 |
| D9 | 3.4 | 2.1 | 1.6 |
| D10 | 3.0 | 1.5 | 0.8 |
| D11 | 0.0 | 1.6 | 0.3 |
| D12 | 3.1 | 1.5 | 0.8 |
| D13 | 3.4 | 0.0 | 0.4 |

For Basic variants, 40° C. at 4 weeks was used since the highest value was noted at that time point.

### Example 7—Total Score Results

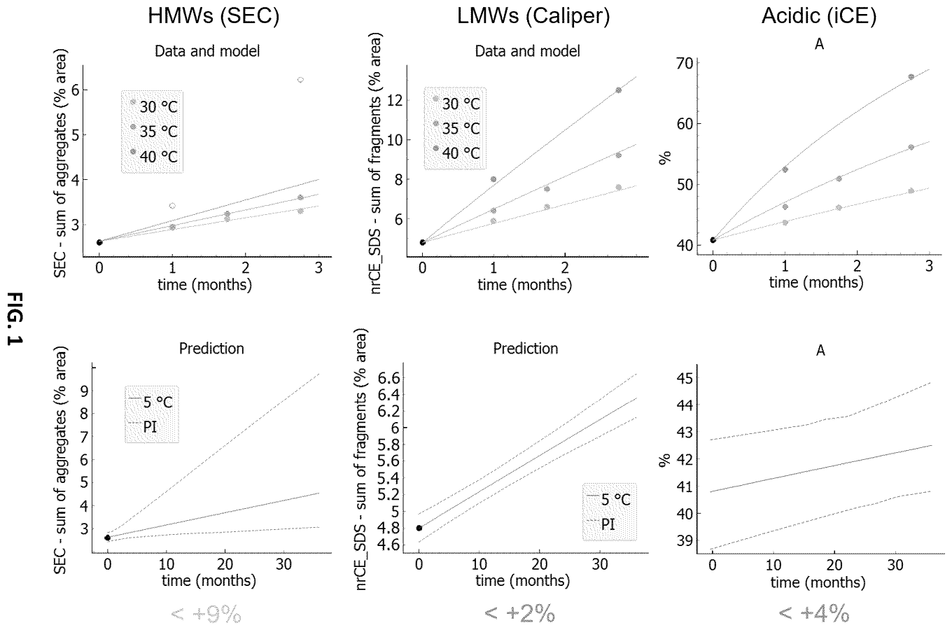

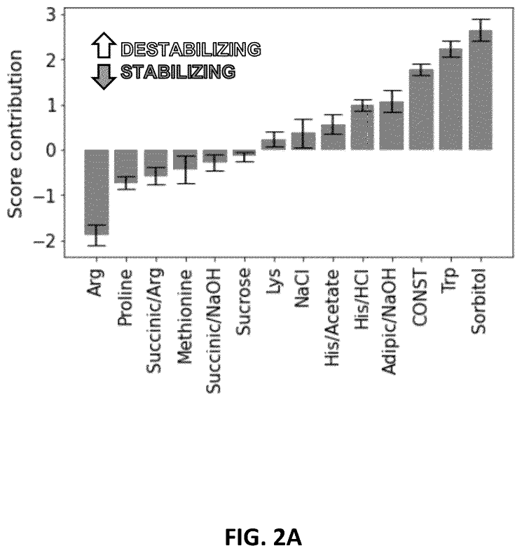

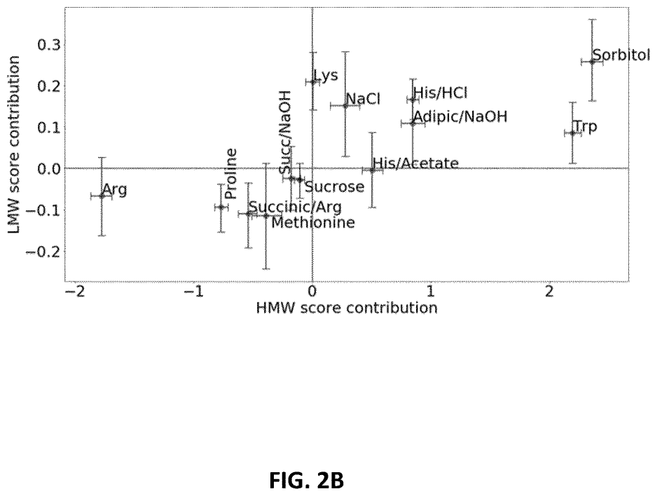

The measurements obtained from the storage conditions (Table 7-Study setup) enable the calculation of an Arrhenius model for long term stability prediction. Since multiple studies were conducted at 40° C., a single scoring based on Arrhenius prediction could be established. For each protein modification attribute (e.g., SEC HMWs), a prediction for 3 years at 5° C. could be made (with 95% confidence interval) for the reference formulation (FIG. 1). With respect to HMWs, the 40° C. data was excluded from the fit due to an apparent switch in the aggregation mechanism. From this, expected values at 5° C. can be linearly extrapolated for other formulations based on their 40° C. measurements. This value is compared to the variability of reference formulations (i.e., originator batches) from several batches with varying shelf-life in a way that bigger ratios (e.g. larger SEC HMWs increase as compared to variability of originator HMWs values) are given a larger score. Formulations are scored for each modification/method and the scores are added up to generate a final score. Basic variants (iCE analysis) were excluded from the score due to poor Arrhenius model fit. Method variabilities are taken into consideration in score error. Linear regression is used for calculation of score contributions of different buffers/excipients. The final score (Table 8) represents overall change/degradation after 3 years on 5° C. compared to variability of originator batches. The excipients can be interpreted as stabilizing (negative coefficients) or destabilizing (positive coefficients) (FIGS. 2A-2B).

### Excipient Contributions

Partial scores for individual methods are calculated according to the formula:

$${S}_{M}^{i} = \frac{Δ {M}_{40 ∘ C}^{i}}{Δ {M}_{40 ∘ C}^{Ref}} \frac{Δ {M}_{5 ∘ C}^{Arr}}{{σ}_{orig}^{QTPP}} ,$$

where the first fraction represents the ratio of attribute changes (after conditions—t₀) measured by method M (e.g., SEC HMWs) between the i-th sample and reference sample as measured at 40° C., the second fraction represents the ratio between projected attribute change of reference sample (Arrhenius prediction, 3y, 95% confidence) and variability of 5 measured originator batches.

The above score represents a weighted result of the

$$Δ {M}_{40 ∘ C}^{i}$$

measurement, with the weight expressed as:

$${w}_{M} = \frac{Δ {M}_{5 ∘ C}^{Arr}}{Δ {M}_{40 ∘ C}^{Ref} {σ}_{orig}^{QTPP}} .$$

The complete score of the i-th sample is the sum of individual method scores divided by the sum of weights:

$${S}^{i} = \frac{{\sum }_{M} {w}_{M} Δ {M}_{40 ∘ C}^{i}}{{\sum }_{M} {w}_{M}} = \frac{{\sum }_{M} {S}_{M}^{i}}{{\sum }_{M} {w}_{M}} .$$

Only methods that showed significant differences were included in the summation.

Linear regression on

$${}_{TOTAL}^{i}$$

results is used for calculation of score contributions of different buffers/excipients (FIG. 1). The score represents overall change/degradation after 3 years on 5° C. compared to variability of originator batches.

TABLE 7
Study setup - Formulations D1-D13

| Storage | | | | |
|---|---|---|---|---|
| Condition | SEC | Nr-CESDS | iCE | UV |
| t0 | X | X | X | X |
| 40° C., 4 weeks | | | X | |
| 40° C., 11 weeks | X | X | X | |

Note:
Additional pull points for three Arrhenius formulations: 30° C. and 35° C., 4 weeks, 7 weeks and 11 weeks.

TABLE 8
Total score, formulations D1-D13, after 3 years on 5° C.

| Composition | Score |
|---|---|
| D1 | 100 |
| D2 | 0 |
| D3 | 100 |
| D4 | 100 |
| D5 | 500 |
| D6 | 900 |
| D7 | 1050 |
| D8 | 700 |
| D9 | 650 |
| D10 | 350 |
| D11 | 1050 |
| D12 | 250 |
| D13 | 250 |

The eTREADS results for compositions DP1_PPM1-DP6_PPM1, including Dupixent (Lot EU_#0L254C-175 mg/ml), (Table 2) after 3 months at 40° C. are presented in Table 9. Formation of aggregates is the highest in the Dupixent sample, DP1_PPM1 (reference material-like formulation) and DP4_PPM1. The increase of acidic variants (acidic peaks) is the highest in the DP1 PPM1 formulation. The number of sub-visible particles is increased in all formulations (except DP6_PPM1); the number of sub-visible particles is lower in all DP formulations than for the Dupixient formulation.

The eTREADS results for compositions DP7_PPM3-DP10_PPM3, including Dupixent (Lot EU_#0L741C-175 mg/mL) (Table 2), after 3 months at 40° C. are presented in Table 10. Formation of aggregates was highest in DP10_PPM3 and the Dupixent sample. All formulations showed lower a lower sum of aggregates and lower amount of particles of all sizes tested than the Dupixent sample.

The eTREADS results for compositions DP1_PPM1-DP6_PPM1, including Dupixent (Lot EU_#0L254C-175 mg/mL), (Table 2) after 3 months at 25° C. is presented in Table 11. The different formulations were fairly comparable with respect to the amount of aggregates (all formulations DP1_PPM1-DP6_PPM1 showed a lower sum of aggregates than the Dupixent reference). All formulations DP1_PPM1-DP6_PPM1 showed fewer observed sub-visible particles than the Dupixent reference. Stability results for compositions DP1_PPM1-DP10_PPM3 after 6 months at 25° C. are shown in Table 12.

The eTREADS results for compositions DP7_PPM3-DP10_PPM3, including Dupixent (Lot EU_#0L741C-175 mg/mL) (Table 2), after 3 months at 25° C. are presented in Table 13. The formulations were similar with respect to the amount of aggregates, fragments or acidic peaks. The highest number of sub-visible particles was highest in DP9_PPM4 and the Dupixent sample.

Results for DP1_PPM1-DP10_PPM3 after 6 months at 5° C. is presented in Table 14.

TABLE 9
eTREADS Results - Thermal stress, 3 months, 40° C.

| | Name of analytical method | | | | | | | | | | |
|---|---|---|---|---|---|---|---|---|---|---|---|
| | SEC | | ICE | | | nrCE-SDS | | MFI | | | |
| | Type of assessment for selected category | | | | | | | | | | |
| | Type 5 | Type 6 | Type 5 | Type 6 | Type 5 | Type 5 | Type 6 | Type 1 | Type 1 | Type 1 | Type 1 |
| | Sum of | Main | Sum of | Sum of | Sum of | Sum of | Purity | Particles >2 | Particles >5 | Particles >10 | Particles >25 |
| Category name: | aggregates | peak | AP | MP | BV | LMW | (MP) | um | um | um | um |
| DP1_PPM1 | 6.0 | 4.6 | 3.6 | 2.4 | 0.1 | 1.7 | 1.4 | 28.1 | 15.8 | 17.7 | 6.9 |
| DP2_PPM1 | 4.0 | 2.8 | 2.1 | 1.9 | 1.1 | 2.0 | 1.6 | 2.1 | 5.2 | 6.7 | 3.5 |
| DP3_PPM1 | 1.5 | 0.0 | 2.7 | 1.5 | 0.0 | 0.4 | 0.3 | 33.2 | 19.1 | 22.3 | 1.7 |
| DP3_PPM2 | 1.6 | 0.0 | 2.9 | 1.6 | 0.0 | 0.4 | 0.3 | 6.8 | 3.7 | 0.0 | 0.2 |
| DP4_PPM1 | 7.6 | 5.1 | 2.0 | 1.9 | 1.1 | 1.3 | 0.9 | 14.0 | 9.4 | 13.0 | 8.0 |
| DP5_PPM1 | 1.5 | 1.1 | 2.7 | 1.6 | 0.2 | 0.0 | 0.0 | 38.0 | 19.5 | 27.3 | 13.9 |
| DP6_PPM1 | 0.0 | 0.0 | 2.7 | 1.5 | 0.0 | 0.4 | 0.3 | 0.0 | 0.0 | 1.0 | 2.2 |
| Dupixent | 16.2 | 4.5 | 0.0 | 0.0 | 1.2 | 0.6 | 1.4 | 92.3 | 48.8 | 34.3 | 0.0 |
| [(EU_#0L254C) | | | | | | | | | | | |

AP—acidic peaks;
MP—main peaks;
BV—basic variants;
LMW—low molecular weight (25 kDa-150 kDa)

TABLE 10
eTREADS Results - Thermal stress, 3 months, 40° C.

| | Name of analytical method | | | | | | | | | | |
|---|---|---|---|---|---|---|---|---|---|---|---|
| | SEC | | ICE | | | nrCE-SDS | | MFI | | | |
| | Type of assessment for selected category | | | | | | | | | | |
| | Type 5 | Type 6 | Type 5 | Type 6 | Type 5 | Type 5 | Type 6 | Type 1 | Type 1 | Type 1 | Type 1 |
| | Sum of | Main | Sum of | Sum of | Sum of | Sum of | Purity | Particles >2 | Particles >5 | Particles >10 | Particles >25 |
| Category name: | aggregates | peak | AP | MP | BV | LMW | (MP) | um | um | um | um |
| DP7_PPM3 | 2.0 | 3.0 | 3.7 | 2.8 | 0.6 | 1.2 | 1.5 | 31.8 | 13.1 | 10.6 | 1.9 |
| DP8_PPM3 | 0.2 | 0.0 | 3.7 | 2.5 | 0.3 | 0.0 | 0.0 | 105.4 | 23.9 | 3.8 | 0.1 |
| DP9_PPM3 | 0.0 | 0.2 | 3.4 | 1.8 | 0.0 | 0.2 | 0.0 | 7.8 | 3.8 | 3.4 | 0.4 |
| DP9_PPM4 | 0.1 | 0.5 | 3.2 | 2.0 | 0.3 | 0.5 | 0.3 | 34.4 | 11.4 | 3.2 | 0.0 |
| DP10_PPM3 | 6.0 | 4.4 | 2.4 | 1.9 | 1.0 | 0.5 | 0.5 | 0.0 | 0.0 | 0.0 | 0.3 |
| Dupixent | 16.0 | 5.7 | 0.0 | 0.0 | 1.5 | 1.0 | 2.0 | 131.9 | 77.6 | 97.4 | 13.1 |
| (EU_#0L741C) | | | | | | | | | | | |

TABLE 11
eTREADS results - Thermal stress after 3 months at 25° C.

| | Name of analytical method | | | | | | | | | | |
|---|---|---|---|---|---|---|---|---|---|---|---|
| | SEC | | ICE | | | nrCE-SDS | | MFI | | | |
| | Type of assessment for selected category | | | | | | | | | | |
| | Type 5 | Type 6 | Type 5 | Type 6 | Type 5 | Type 5 | Type 6 | Type 1 | Type 1 | Type 1 | Type 1 |
| | Sum of | Main | Sum of | Sum of | Sum of | Sum of | Purity | Particles >2 | Particles >5 | Particles >10 | Particles >25 |
| Category name: | aggregates | peak | AP | MP | BV | LMW | (MP) | um | um | um | um |
| DP1_PPM1 | 0.2 | 0.3 | 0.3 | 0.3 | 0.0 | 0.0 | 0.0 | 9.4 | 0.8 | 0.0 | 0.4 |
| DP2_PPM1 | 0.2 | 1.4 | 0.4 | 0.7 | 0.3 | 0.0 | 0.0 | 0.0 | 0.0 | 0.1 | 0.0 |
| DP3_PPM1 | 0.6 | 1.4 | 0.1 | 0.2 | 0.1 | 0.1 | 0.2 | 8.1 | 2.9 | 2.2 | 0.0 |
| DP3_PPM2 | 0.2 | 1.2 | 0.0 | 0.0 | 0.0 | 0.0 | 0.0 | 14.9 | 2.9 | 1.4 | 0.4 |
| DP4_PPM1 | 0.0 | 0.5 | 0.2 | 0.4 | 0.3 | 0.0 | 0.1 | 0.4 | 0.1 | 0.5 | 0.4 |
| DP5_PPM1 | 0.9 | 1.1 | 0.2 | 0.2 | 0.0 | 0.1 | 0.2 | 13.1 | 3.7 | 2.0 | 0.0 |
| DP6_PPM1 | 0.6 | 1.6 | 0.1 | 0.0 | 0.0 | 0.0 | 0.2 | 7.2 | 1.1 | 2.9 | 2.3 |
| Dupixent | 3.3 | 0.0 | 0.1 | 0.3 | 0.3 | 0.0 | 0.7 | 46.3 | 17.0 | 17.9 | 1.8 |
| (EU_#0L254C) | | | | | | | | | | | |

TABLE 12
eTREADS Results - Thermal stress, 6 months, 25° C.

| | Name of analytical method | | | | | | | | | | |
|---|---|---|---|---|---|---|---|---|---|---|---|
| | SEC | | ICE | | | nrCE-SDS | | MFI | | | |
| | Type of assessment for selected category | | | | | | | | | | |
| | Type 5 | Type 6 | Type 5 | Type 6 | Type 5 | Type 5 | Type 6 | Type 1 | Type 1 | Type 1 | Type 1 |
| | Sum of | Main | Sum of | Sum of | Sum of | Sum of | Purity | Particles >2 | Particles >5 | Particles >10 | Particles >25 |
| Category name: | aggregates | peak | AP | MP | BV | LMW | (MP) | um | um | um | um |
| DP1_PPM1 | 1.4 | 1.1 | 0.6 | 1.0 | 0.5 | 0.2 | 0.2 | 0.0 | 0.0 | 0.0 | 0.4 |
| DP2_PPM1 | 0.0 | 0.6 | 0.2 | 1.2 | 1.1 | 0.1 | 0.1 | 0.2 | 0.4 | 0.8 | 1.7 |
| DP3_PPM1 | 0.7 | 1.2 | 0.2 | 0.2 | 0.0 | 0.2 | 0.4 | 2.1 | 1.3 | 0.7 | 1.0 |
| DP3_PPM2 | 0.7 | 0.7 | 0.3 | 0.5 | 0.3 | 0.0 | 0.4 | 7.7 | 4.1 | 3.1 | 1.7 |
| DP4_PPM1 | 0.1 | 0.5 | 0.1 | 0.8 | 0.8 | 0.1 | 0.2 | 12.5 | 9.2 | 5.9 | 3.4 |
| DP5_PPM1 | 1.7 | 0.7 | 0.2 | 0.7 | 0.7 | 0.1 | 0.2 | 32.3 | 7.9 | 3.2 | 2.8 |
| DP6_PPM1 | 0.6 | 0.4 | 0.3 | 0.7 | 0.6 | 0.5 | 0.5 | 8.0 | 3.0 | 3.9 | 4.6 |
| DP7_PPM3 | 1.1 | 1.2 | 0.5 | 0.9 | 0.5 | 0.4 | 0.4 | 53.7 | 13.2 | 3.9 | 0.2 |
| DP8_PPM3 | — | 0.0 | 0.1 | 0.3 | 0.3 | 0.2 | 0.2 | 25.8 | 5.2 | 0.2 | 0.0 |
| DP9_PPM3 | 1.1 | 0.7 | 0.1 | 0.0 | 0.0 | 0.1 | 0.0 | 14.2 | 3.0 | 1.4 | 0.4 |
| DP9_PPM4 | 1.6 | 0.9 | 0.0 | 0.0 | 0.0 | 0.2 | 0.4 | 5.6 | 2.3 | 1.8 | 0.0 |
| DP10_PPM3 | 0.8 | 0.3 | 0.0 | 0.2 | 0.3 | 0.6 | 0.4 | 8.7 | 1.6 | 0.0 | 0.0 |

TABLE 13
eTREADS Results - Thermal stress, 3 months, 25° C.

| | Name of analytical method | | | | | | | | | | |
|---|---|---|---|---|---|---|---|---|---|---|---|
| | SEC | | ICE | | | nrCE-SDS | | MFI | | | |
| | Type of assessment for selected category | | | | | | | | | | |
| | Type 5 | Type 6 | Type 5 | Type 6 | Type 5 | Type 5 | Type 6 | Type 1 | Type 1 | Type 1 | Type 1 |
| | Sum of | Main | Sum of | Sum of | Sum of | Sum of | Purity | Particles >2 | Particles >5 | Particles >10 | Particles >25 |
| Category name: | aggregates | peak | AP | MP | BV | LMW | (MP) | um | um | um | um |
| DP7_PPM3 | 1.1 | 1.1 | 0.4 | 0.7 | 0.4 | 0.2 | 0.2 | 14.4 | 3.0 | 0.2 | 0.0 |
| DP8_PPM3 | 0.7 | 0.1 | 0.1 | 0.1 | 0.2 | 0.2 | 0.1 | 2.4 | 0.4 | 0.0 | 0.2 |
| DP9_PPM3 | 1.2 | 1.3 | 0.2 | 0.0 | 0.0 | 0.1 | 0.0 | 0.0 | 0.0 | 1.3 | 0.2 |
| DP9_PPM4 | 1.6 | 1.8 | 0.0 | 0.0 | 0.1 | 0.2 | 0.2 | 37.7 | 12.0 | 9.4 | 2.3 |
| DP10_PPM3 | 0.9 | 1.1 | 0.3 | 0.2 | 0.1 | 0.4 | 0.2 | 19.9 | 3.7 | 0.5 | 0.0 |
| Dupixent | 0.0 | 0.0 | 0.0 | 0.1 | 0.3 | 0.0 | 0.3 | 41.9 | 16.8 | 17.8 | 1.8 |
| (EU_#0L741C) | | | | | | | | | | | |

TABLE 14
eTREADS Results - Thermal stress, 6 months, 5° C.

| | Name of analytical method | | | | | | | | | | |
|---|---|---|---|---|---|---|---|---|---|---|---|
| | SEC | | ICE | | | nrCE-SDS | | MFI | | | |
| | Type of assessment for selected category | | | | | | | | | | |
| | Type 5 | Type 6 | Type 5 | Type 6 | Type 5 | Type 5 | Type 6 | Type 1 | Type 1 | Type 1 | Type 1 |
| | Sum of | Main | Sum of | Sum of | Sum of | Sum of | Purity | Particles >2 | Particles >5 | Particles >10 | Particles >25 |
| Category name: | aggregates | peak | AP | MP | BV | LMW | (MP) | um | um | um | um |
| DP1_PPM1 | 0.1 | 0.0 | 0.1 | 0.1 | 0.0 | 0.0 | 0.0 | 1.9 | 0.6 | 1.6 | 0.7 |
| DP2_PPM1 | 0.3 | 0.0 | 0.1 | 0.1 | 0.0 | 0.0 | 0.1 | 0.0 | 0.0 | 0.0 | 0.2 |
| DP3_PPM1 | 0.6 | 0.0 | 0.0 | 0.0 | 0.0 | 0.0 | 0.2 | 0.3 | 0.2 | 0.9 | 0.0 |
| DP3_PPM2 | 0.1 | 0.0 | 0.3 | 0.4 | 0.1 | 0.0 | 0.0 | 2.5 | 1.1 | 1.5 | 0.9 |
| DP4_PPM1 | 0.1 | 0.0 | 0.5 | 0.8 | 0.3 | 0.0 | 0.0 | 8.3 | 2.1 | 1.8 | 1.2 |
| DP5_PPM1 | 0.8 | 0.1 | 0.2 | 0.4 | 0.3 | 0.0 | 0.0 | 1.1 | 0.1 | 0.3 | 1.4 |
| DP6_PPM1 | 0.0 | 0.4 | 0.6 | 0.5 | 0.0 | 0.0 | 0.1 | 2.3 | 1.6 | 3.9 | 4.0 |
| DP7_PPM3 | 0.8 | 0.0 | 0.6 | 1.0 | 0.3 | 0.0 | 0.0 | 4.0 | 1.5 | 1.8 | 1.7 |
| DP8_PPM3 | 0.0 | 0.0 | 0.5 | 0.8 | 0.3 | 0.0 | 0.0 | 4.7 | 0.5 | 0.2 | 0.2 |
| DP9_PPM3 | 0.4 | 0.0 | 0.3 | 0.3 | 0.0 | 0.0 | 0.0 | 6.4 | 1.1 | 1.1 | 0.9 |
| DP9_PPM4 | 0.8 | 0.0 | 0.3 | 0.9 | 0.6 | 0.0 | 0.1 | 16.1 | 8.1 | 7.6 | 1.9 |
| DP10_PPM3 | 0.9 | 0.1 | 0.3 | 0.7 | 0.4 | 0.0 | 0.0 | 36.6 | 30.1 | 24.4 | 0.0 |
| Dupixent | 0.5 | 0.0 | 0.3 | 0.5 | 0.3 | 0.0 | 0.0 | 0.8 | 0.0 | 0.0 | 0.9 |
| (EU_#0L741C) | | | | | | | | | | | |
| Dupixent | 0.0 | 0.0 | 0.2 | 0.7 | 0.4 | 0.0 | 0.1 | 40.9 | 25.4 | 21.4 | 0.9 |
| (EU_#0L254C) | | | | | | | | | | | |

The eTREADS results for compositions DP1_PPM1-DP6_PPM1 (Table 2) after mechanical stress and 2.5 months at 25° C. are presented in Table 15. The eTREADS results for compositions DP7_PPM3-DP10_PPM3 (Table 2) after mechanical stress and 2.5 months at 25° C. are presented in Table 16.

The eTREADS results for compositions DP1_PPM1-DP6_PPM1, including Dupixent (Lot EU_#0L254C-175 mg/mL), (Table 2) after light exposure and 2.5 months at 25° C. is presented in Table 16. The eTREADS results for compositions DP7_PPM3-DP10_PPM3, including Dupixent (Lot EU_#0L741C-175 mg/mL), (Table 2) after light exposure and 2.5 months at 25° C. is presented in Table 17.

TABLE 15
eTREADS results after mechanical stress and 2.5 months at 25° C.

| | Name of analytical method | | | | | | | |
|---|---|---|---|---|---|---|---|---|
| | SEC | | nrCE-SDS | | MFI | | | |
| | Type of assessment for selected category | | | | | | | |
| | Type 5 | Type 6 | Type 5 | Type 6 | Type 1 | Type 1 | Type 1 | Type 1 |
| | Sum of | Main | Sum of | Purity | Particles >2 | Particles >5 | Particles >10 | Particles >25 |
| Category name: | aggregates | peak | LMW | (MP) | um | um | um | um |
| DP1_PPM1 | 0.8 | 1.2 | 0.0 | 0.1 | 33.4 | 11.1 | 6.7 | 0.2 |
| DP2_PPM1 | 0.0 | 0.7 | 0.0 | 0.2 | 0.0 | 0.0 | 0.0 | 0.4 |
| DP3_PPM1 | 0.3 | 1.1 | 0.1 | 0.2 | 51.7 | 14.1 | 5.5 | 0.0 |
| DP3_PPM2 | 0.0 | 0.4 | 0.0 | 0.1 | 18.3 | 6.0 | 3.2 | 0.9 |
| DP4_PPM1 | 0.0 | 0.0 | 0.0 | 0.0 | 2.8 | 1.1 | 0.0 | 0.4 |
| DP5_PPM1 | 1.8 | 2.2 | 0.0 | 0.1 | 9.6 | 4.0 | 3.2 | 1.5 |
| DP6_PPM1 | 0.3 | 1.1 | 0.0 | 0.1 | 6.7 | 3.3 | 7.1 | 5.1 |

TABLE 16
eTREADS results after mechanical stress and 2.5 months at 25° C.

| | Name of analytical method | | | | | | | |
|---|---|---|---|---|---|---|---|---|
| | SEC | | nrCE-SDS | | MFI | | | |
| | Type of assessment for selected category | | | | | | | |
| | Type 5 | Type 6 | Type 5 | Type 6 | Type 1 | Type 1 | Type 1 | Type 1 |
| | Sum of | Main | Sum of | Purity | Particles >2 | Particles >5 | Particles >10 | Particles >25 |
| Category name: | aggregates | peak | LMW | (MP) | um | um | um | um |
| DP7_PPM3 | 0.4 | 0.4 | 0.2 | 0.4 | 29.4 | 11.5 | 3.4 | 0.0 |
| DP8_PPM3 | 0.0 | 0.0 | 0.2 | 0.3 | 17.5 | 6.5 | 1.6 | 0.2 |
| DP9_PPM3 | 0.0 | 0.3 | 0.0 | 0.0 | 1.8 | 0.1 | 0.7 | 0.0 |
| DP9_PPM4 | 0.1 | 0.9 | 0.4 | 0.5 | 0.0 | 9.7 | 22.5 | 2.6 |
| DP10_PPM3 | 0.0 | 0.8 | 0.2 | 0.4 | 3.2 | 0.0 | 0.0 | 0.2 |

TABLE 16
eTREADS results after Light Exposure and 2.5 months at 25° C.

| | Name of analytical method | | | | | | | | | | |
|---|---|---|---|---|---|---|---|---|---|---|---|
| | SEC | | ICE | | | nrCE-SDS | | MFI | | | |
| | Type of assessment for selected category | | | | | | | | | | |
| | Type 5 | Type 6 | Type 5 | Type 6 | Type 5 | Type 5 | Type 6 | Type 1 | Type 1 | Type 1 | Type 1 |
| | Sum of | Main | Sum of | Sum of | Sum of | Sum of | Purity | Particles >2 | Particles >5 | Particles >10 | Particles >25 |
| Category name: | aggregates | peak | AP | MP | BV | LMW | (MP) | um | um | um | um |
| DP1_PPM1 | 1.5 | 0.3 | 0.5 | 0.3 | 0.0 | 0.9 | 0.0 | 7.2 | 1.0 | 0.3 | 0.1 |
| DP2_PPM1 | 0.9 | 0.1 | 0.2 | 0.3 | 0.2 | 1.9 | 0.5 | 0.0 | 1.2 | 1.5 | 0.6 |
| DP3_PPM1 | 2.6 | 0.6 | 0.0 | 0.0 | 0.2 | 1.6 | 0.7 | 2.2 | 1.4 | 0.5 | 0.0 |
| DP3_PPM2 | 3.8 | 2.0 | 0.3 | 0.4 | 0.2 | 1.0 | 0.4 | 20.8 | 5.9 | 2.2 | 1.2 |
| DP4_PPM1 | 0.0 | 0.0 | 0.1 | 0.1 | 0.0 | 1.0 | 0.3 | 2.2 | 0.0 | 0.0 | 0.0 |
| DP5_PPM1 | 3.1 | 2.2 | 0.0 | 0.0 | 0.0 | 1.0 | 0.5 | 28.5 | 7.7 | 6.5 | 0.7 |
| DP6_PPM1 | 1.2 | 0.2 | 0.2 | 0.0 | 0.0 | 0.8 | 0.3 | 6.7 | 1.6 | 7.1 | 4.1 |
| Dupixent | 6.2 | 4.2 | 0.0 | 0.0 | 0.1 | 0.0 | 0.3 | 31.1 | 10.1 | 11.7 | 1.5 |
| (EU_#0L254C) | | | | | | | | | | | |

TABLE 17
eTREADS results after Light Exposure and 2.5 months at 25° C.

| | Name of analytical method | | | | | | | | | | |
|---|---|---|---|---|---|---|---|---|---|---|---|
| | SEC | | ICE | | | nrCE-SDS | | MFI | | | |
| | Type of assessment for selected category | | | | | | | | | | |
| | Type 5 | Type 6 | Type 5 | Type 6 | Type 5 | Type 5 | Type 6 | Type 1 | Type 1 | Type 1 | Type 1 |
| | Sum of | Main | Sum of | Sum of | Sum of | Sum of | Purity | Particles >2 | Particles >5 | Particles >10 | Particles >25 |
| Category name: | aggregates | peak | AP | MP | BV | LMW | (MP) | um | um | um | um |
| DP7_PPM3 | 0.0 | 0.0 | 0.6 | 0.6 | 0.2 | 1.2 | 0.3 | 23.2 | 6.6 | 3.5 | 0.0 |
| DP8_PPM3 | 1.3 | 0.4 | 0.4 | 0.5 | 0.4 | 1.4 | 0.7 | 14.8 | 6.3 | 2.4 | 0.4 |
| DP9_PPM3 | 1.0 | 0.3 | 0.1 | 0.0 | 0.0 | 0.9 | 0.0 | 0.0 | 1.1 | 1.0 | 0.2 |
| DP9_PPM4 | 2.1 | 0.9 | 0.0 | 0.1 | 0.3 | 1.2 | 0.5 | 43.1 | 26.3 | 23.6 | 1.2 |
| DP10_PPM3 | 0.0 | 0.0 | 0.2 | 0.0 | 0.0 | 1.0 | 0.1 | 0.2 | 0.0 | 0.0 | 0.0 |
| Dupixent | 6.3 | 4.9 | 0.5 | 0.5 | 0.2 | 0.0 | 0.4 | 17.4 | 10.1 | 4.9 | 0.0 |
| (EU_#0L741C) | | | | | | | | | | | |

TABLE 18
Placebos after mechanical stress and 2.5 months at 25° C.

MFI - Sum of subvisible particles per mL

| | >2 um | >5 um | >10 um | >25 um |
|---|---|---|---|---|
| P1 PPM3 | 14622 | 949 | 20 | 2 |
| P1 PPM4 | 100925 | 12726 | 1547 | 223 |
| P2 PPM3 | 17713 | 828 | 62 | 3 |
| P2 PPM4 | 112083 | 10649 | 1344 | 129 |
| P3 PPM3 | 27576 | 626 | 11 | 3 |
| P3 PPM4 | 71053 | 10278 | 1033 | 119 |
| P4 PPM3 | 28414 | 695 | 15 | 2 |
| P4 PPM4 | 74838 | 3588 | 472 | 33 |

As shown in Table 19, thermal stress did not have any impact on BLI results.

TABLE 19
BLI results after thermal stress (40° C., 3 months)

| | 40° C., 3 months |
|---|---|
| | BLI_FcRn - Norm |
| | Average 1/KD [%] |
| DP2 PPM1 | 101.6 |
| DP3 PPM1 | — |
| DP3 PPM2 | 100.5 |
| DP4 PPM1 | 100.2 |
| DP8 PPM3 | 100.5 |
| DP9 PPM3 | — |
| DP9 PPM4 | 101.4 |
| DP10 PPM3 | 101.3 |
| Dupixent | 98.0 |
| (EU_#OL741C) | |
| Dupixent | 99.5 |
| (EU_#OL254C) | |

Other high protein concentration formulations representing various buffers, and other components are described in Table 20.

TABLE 20

| Protein | | Buffer | | | | | | Surfactant |
|---|---|---|---|---|---|---|---|---|
| conc. | | conc. | | conc. | | conc | | conc. |
| [mg/mL] | Buffer | [mM] | Component | [mM] | Component | [mM] | Surfactant | [mg/mL] |
| 150 | His/HCl | 10 | proline | 150 | Arg HCl | 50 | PS80 | 2 |
| 175 | His/HCl | 10 | proline | 150 | Arg HCl | 50 | PS80 | 2 |
| 150 | His/HCl | 10 | — | — | Arg HCl | 150 | PS80 | 2 |
| 175 | His/HCl | 10 | — | — | Arg HCl | 150 | PS80 | 2 |
| 150 | His/HCl | 20 | — | — | Arg HCl | 150 | PS80 | 2 |
| 175 | His/HCl | 20 | — | — | Arg HCl | 150 | PS80 | 2 |
| 150 | Succinic | 10 | Proline | 150 | Arg HCl | 50 | PS80 | 2 |
| | acid/NaOH | | | | | | | |
| 175 | Succinic | 10 | Proline | 150 | Arg HCl | 50 | PS80 | 2 |
| | acid/NaOH | | | | | | | |
| 150 | Succinic | 10 | Proline/Methionine | 150/10 | Arg HCl | 50 | PS80 | 2 |
| | acid/NaOH | | | | | | | |
| 175 | Succinic | 10 | Proline/Methionine | 150/10 | Arg HCl | 50 | PS80 | 2 |
| | acid/NaOH | | | | | | | |
| 150 | — | — | Sucrose | 150 | Arg HCl | 50 | PS80 | 2 |
| 175 | — | — | Sucrose | 150 | Arg HCl | 50 | PS80 | 2 |
| 150 or | His/Ac | 10 | sucrose | 150 | Arg HCl | 25 | PS80 | 2 |
| 175 | | | | | | | | |
| 150 or | His/Ac | 10 | sucrose | 150 | Arg HCl | 50 | PS80 | 2 |
| 175 | | | | | | | | |
| 150 or | His/Ac | 10 | proline | 150 | Arg HCl | 25 | PS80 | 2 |
| 175 | | | | | | | | |
| 150 or | His/Ac | 10 | proline | 150 | Arg HCl | 50 | PS80 | 2 |
| 175 | | | | | | | | |
| 150 or | His/Ac | 10 | methionine | 150 | Arg HCl | 25 | PS80 | 2 |
| 175 | | | | | | | | |
| 150 or | His/Ac | 10 | methionine | 150 | Arg HCl | 50 | PS80 | 2 |
| 175 | | | | | | | | |
| 150 or | His/Ac | 10 | Sucrose/methionine | 150/10 | Arg HCl | 25 | PS80 | 2 |
| 175 | | | | | | | | |
| 150 or | His/Ac | 10 | Sucrose/methionine | 150/10 | Arg HCl | 50 | PS80 | 2 |
| 175 | | | | | | | | |
| 150 or | His/Ac | 10 | Sucrose/proline | 150/10 | Arg HCl | 25 | PS80 | 2 |
| 175 | | | | | | | | |
| 150 or | His/Ac | 10 | Sucrose/proline | 150/10 | Arg HCl | 50 | PS80 | 2 |
| 175 | | | | | | | | |
| 150 or | His/Ac | 10 | methionine/proline | 150/10 | Arg HCl | 25 | PS80 | 2 |
| 175 | | | | | | | | |
| 150 or | His/Ac | 10 | methionine/proline | 150/10 | Arg HCl | 50 | PS80 | 2 |
| 175 | | | | | | | | |
| 150 or | His/Ac | 10 | proline/methionine | 150/10 | Arg HCl | 25 | PS80 | 2 |
| 175 | | | | | | | | |
| 150 or | His/Ac | 10 | proline/methionine | 150/10 | Arg HCl | 50 | PS80 | 2 |
| 175 | | | | | | | | |
| 150 or | His/Ac | 20 | proline | 150 | Arg HCl | 25 | PS80 | 2 |
| 175 | | | | | | | | |
| 150 or | His/Ac | 20 | proline | 150 | Arg HCl | 50 | PS80 | 2 |
| 175 | | | | | | | | |
| 150 or | His/Ac | 20 | methionine | 150 | Arg HCl | 25 | PS80 | 2 |
| 175 | | | | | | | | |
| 150 or | His/Ac | 20 | methionine | 150 | Arg HCl | 50 | PS80 | 2 |
| 175 | | | | | | | | |
| 150 or | His/Ac | 20 | Sucrose/methionine | 150/10 | Arg HCl | 25 | PS80 | 2 |
| 175 | | | | | | | | |
| 150 or | His/Ac | 20 | Sucrose/methionine | 150/10 | Arg HCl | 50 | PS80 | 2 |
| 175 | | | | | | | | |
| 150 or | His/Ac | 20 | Sucrose/proline | 150/10 | Arg HCl | 25 | PS80 | 2 |
| 175 | | | | | | | | |
| 150 or | His/Ac | 20 | Sucrose/proline | 150/10 | Arg HCl | 50 | PS80 | 2 |
| 175 | | | | | | | | |
| 150 or | His/Ac | 20 | methionine/proline | 150/10 | Arg HCl | 25 | PS80 | 2 |
| 175 | | | | | | | | |
| 150 or | His/Ac | 20 | methionine/proline | 150/10 | Arg HCl | 50 | PS80 | 2 |
| 175 | | | | | | | | |
| 150 or | His/Ac | 20 | proline/methionine | 150/10 | Arg HCl | 25 | PS80 | 2 |
| 175 | | | | | | | | |
| 150 or | His/Ac | 20 | proline/methionine | 150/10 | Arg HCl | 50 | PS80 | 2 |
| 175 | | | | | | | | |
| 150 or | His/Ac | 10 or | — | — | Arg HCl | 150 | PS80 | 2 |
| 175 | | 20 | | | | | | |
| 150 or | His/HCl | 10 | sucrose | 150 | Arg HCl | 25 | PS80 | 2 |
| 175 | | | | | | | | |
| 150 or | His/HCl | 10 | sucrose | 150 | Arg HCl | 50 | PS80 | 2 |
| 175 | | | | | | | | |
| 150 or | His/HCl | 10 | proline | 150 | Arg HCl | 25 | PS80 | 2 |
| 175 | | | | | | | | |
| 150 or | His/HCl | 10 | methionine | 150 | Arg HCl | 25 | PS80 | 2 |
| 175 | | | | | | | | |
| 150 or | His/HCl | 10 | methionine | 150 | Arg HCl | 50 | PS80 | 2 |
| 175 | | | | | | | | |
| 150 or | His/HCl | 10 | Sucrose/methionine | 150/10 | Arg HCl | 25 | PS80 | 2 |
| 175 | | | | | | | | |
| 150 or | His/HCl | 10 | Sucrose/methionine | 150/10 | Arg HCl | 50 | PS80 | 2 |
| 175 | | | | | | | | |
| 150 or | His/HCl | 10 | Sucrose/proline | 150/10 | Arg HCl | 25 | PS80 | 2 |
| 175 | | | | | | | | |
| 150 or | His/HCl | 10 | Sucrose/proline | 150/10 | Arg HCl | 50 | PS80 | 2 |
| 175 | | | | | | | | |
| 150 or | His/HCl | 10 | methionine/proline | 150/10 | Arg HCl | 25 | PS80 | 2 |
| 175 | | | | | | | | |
| 150 or | His/HCl | 10 | methionine/proline | 150/10 | Arg HCl | 50 | PS80 | 2 |
| 175 | | | | | | | | |
| 150 or | His/HCl | 10 | proline/methionine | 150/10 | Arg HCl | 25 | PS80 | 2 |
| 175 | | | | | | | | |
| 150 or | His/HCl | 10 | proline/methionine | 150/10 | Arg HCl | 50 | PS80 | 2 |
| 175 | | | | | | | | |
| 150 or | His/HCl | 20 | Sucrose | 150 | Arg HCl | 25 | PS80 | 2 |
| 175 | | | | | | | | |
| 150 or | His/HCl | 20 | Sucrose | 150 | Arg HCl | 50 | PS80 | 2 |
| 175 | | | | | | | | |
| 150 or | His/HCl | 20 | proline | 150 | Arg HCl | 25 | PS80 | 2 |
| 175 | | | | | | | | |
| 150 or | His/HCl | 20 | proline | 150 | Arg HCl | 50 | PS80 | 2 |
| 175 | | | | | | | | |
| 150 or | His/HCl | 20 | methionine | 150 | Arg HCl | 25 | PS80 | 2 |
| 175 | | | | | | | | |
| 150 or | His/HCl | 20 | methionine | 150 | Arg HCl | 50 | PS80 | 2 |
| 175 | | | | | | | | |
| 150 or | His/HCl | 20 | Sucrose/methionine | 150/10 | Arg HCl | 25 | PS80 | 2 |
| 175 | | | | | | | | |
| 150 or | His/HCl | 20 | Sucrose/methionine | 150/10 | Arg HCl | 50 | PS80 | 2 |
| 175 | | | | | | | | |
| 150 or | His/HCl | 20 | Sucrose/proline | 150/10 | Arg HCl | 25 | PS80 | 2 |
| 175 | | | | | | | | |
| 150 or | His/HCl | 20 | Sucrose/proline | 150/10 | Arg HCl | 50 | PS80 | 2 |
| 175 | | | | | | | | |
| 150 or | His/HCl | 20 | methionine/proline | 150/10 | Arg HCl | 25 | PS80 | 2 |
| 175 | | | | | | | | |
| 150 or | His/HCl | 20 | methionine/proline | 150/10 | Arg HCl | 50 | PS80 | 2 |
| 175 | | | | | | | | |
| 150 or | His/HCl | 20 | proline/methionine | 150/10 | Arg HCl | 25 | PS80 | 2 |
| 175 | | | | | | | | |
| 150 or | His/HCl | 20 | proline/methionine | 150/10 | Arg HCl | 50 | PS80 | 2 |
| 175 | | | | | | | | |
| 150 or | Succinic | 10 | Sucrose | 150 | Arg HCl | 50 | PS80 | 2 |
| 175 | acid/NaOH | | | | | | | |
| 150 or | Succinic | 10 | methionine | 150 | Arg HCl | 50 | PS80 | 2 |
| 175 | acid/NaOH | | | | | | | |
| 150 or | Succinic | 10 | Sucrose/methionine | 150/10 | Arg HCl | 50 | PS80 | 2 |
| 175 | acid/NaOH | | | | | | | |
| 150 or | Succinic | 10 | Sucrose/proline | 150/10 | Arg HCl | 50 | PS80 | 2 |
| 175 | acid/NaOH | | | | | | | |
| 150 or | Succinic | 10 | Methionine/Proline | 150/10 | Arg HCl | 50 | PS80 | 2 |
| 175 | acid/NaOH | | | | | | | |
| 150 or | Succinic | 20 | Sucrose | 150 | Arg HCl | 50 | PS80 | 2 |
| 175 | acid/NaOH | | | | | | | |
| 150 or | Succinic | 20 | Methionine | 150 | Arg HCl | 50 | PS80 | 2 |
| 175 | acid/NaOH | | | | | | | |
| 150 or | Succinic | 20 | Proline | 150 | Arg HCl | 50 | PS80 | 2 |
| 175 | acid/NaOH | | | | | | | |
| 150 or | Succinic | 20 | Sucrose/Methionine | 150/10 | Arg HCl | 50 | PS80 | 2 |
| 175 | acid/NaOH | | | | | | | |
| 150 or | Succinic | 20 | Sucrose/Proline | 150/10 | Arg HCl | 50 | PS80 | 2 |
| 175 | acid/NaOH | | | | | | | |
| 150 or | Succinic | 20 | Proline/Methionine | 150/10 | Arg HCl | 50 | PS80 | 2 |
| 175 | acid/NaOH | | | | | | | |
| 150 or | Succinic | 20 | Methionine/Proline | 150/10 | Arg HCl | 50 | PS80 | 2 |
| 175 | acid/NaOH | | | | | | | |
| 150 or | Succinic | 10 or | Proline | 150 | Lysine | 75 | PS80 | 2 |
| 175 | acid/NaOH | 20 | | | | | | |
| 150 or | Succinic | 10 or | Methionine | 150 | Lysine | 75 | PS80 | 2 |
| 175 | acid/NaOH | 20 | | | | | | |
| 150 or | Succinic | 10 or | Sucrose | 150 | Lysine | 75 | PS80 | 2 |
| 175 | acid/NaOH | 20 | | | | | | |
| 150 or | Succinic | 10 or | Sucrose/Methionine | 150/10 | Lysine | 75 | PS80 | 2 |
| 175 | acid/NaOH | 20 | | | | | | |
| 150 or | Succinic | 10 or | Sucrose/Proline | 150/10 | Lysine | 75 | PS80 | 2 |
| 175 | acid/NaOH | 20 | | | | | | |
| 150 or | Succinic | 10 or | Methionine/Proline | 150/10 | Lysine | 75 | PS80 | 2 |
| 175 | acid/NaOH | 20 | | | | | | |
| 150 or | Succinic | 10 or | Proline/Methionine | 150/10 | Lysine | 75 | PS80 | 2 |
| 175 | acid/NaOH | 20 | | | | | | |
| 150 or | Succinic | 10 or | Proline/Methionine | 150/10 | Lysine | 75 | PS80 | 2 |
| 175 | acid/NaOH | 20 | | | | | | |
| 150 or | Succinic | 10 or | — | — | Arg HCl | 150 | PS80 | 2 |
| 175 | acid/NaOH | 20 | | | | | | |
| 150 or | Adipic | 10 or | Sucrose | 150 | Arg HCl | 50 | PS80 | 2 |
| 175 | acid/NaOH | 20 | | | | | | |
| 150 or | Adipic | 10 or | Methionine | 150 | Arg HCl | 50 | PS80 | 2 |
| 175 | acid/NaOH | 20 | | | | | | |
| 150 or | Adipic | 10 or | Proline | 150 | Arg HCl | 50 | PS80 | 2 |
| 175 | acid/NaOH | 20 | | | | | | |
| 150 or | Adipic | 10 or | Sucrose/Methionine | 150/10 | Arg HCl | 50 | PS80 | 2 |
| 175 | acid/NaOH | 20 | | | | | | |
| 150 or | Adipic | 10 or | Sucrose/Proline | 150/10 | Arg HCl | 50 | PS80 | 2 |
| 175 | acid/NaOH | 20 | | | | | | |
| 150 or | Adipic | 10 or | Methionine/Proline | 150/10 | Arg HCl | 50 | PS80 | 2 |
| 175 | acid/NaOH | 20 | | | | | | |
| 150 or | Adipic | 10 or | Proline/Methionine | 150/10 | Arg HCl | 50 | PS80 | 2 |
| 175 | acid/NaOH | 20 | | | | | | |
| 150 or | Adipic | 10 or | — | — | Arg HCl | 150 | PS80 | 2 |
| 175 | acid/NaOH | 20 | | | | | | |
| 150 or | — | — | Proline | 150 | Arg HCl | 50 | PS80 | 2 |
| 175 | | | | | | | | |
| 150 or | — | — | Methionine | 150 | Arg HCl | 50 | PS80 | 2 |
| 175 | | | | | | | | |
| 150 or | — | — | Sucrose/Methionine | 150/10 | Arg HCl | 50 | PS80 | 2 |
| 175 | | | | | | | | |
| 150 or | — | — | Sucrose/Proline | 150/10 | Arg HCl | 50 | PS80 | 2 |
| 175 | | | | | | | | |
| 150 or | — | — | Methionine/Proline | 150/10 | Arg HCl | 50 | PS80 | 2 |
| 175 | | | | | | | | |
| 150 or | — | — | Proline/Methionine | 150/10 | Arg HCl | 50 | PS80 | 2 |
| 175 | | | | | | | | |

His/Ace: histidine/acetate;
Arg: L-arginine hydrochloride;
His/HCl: Histidine/HCl;
PS80: polysorbate 80;
All compositions are pH 5.9.

Although various embodiments of the invention have been described using specific terms, devices, and methods, such description is for illustrative purposes only. The words used are words of description rather than of limitation. It is to be understood that changes and variations may be made by those skilled in the art without departing from the spirit or scope of the present invention, which is set forth in the following claims. In addition, it should be understood that aspects of the various embodiments may be interchanged either in whole or in part. Therefore, the spirit and scope of the appended claims should not be limited to the description of the preferred versions contained therein.

1. A composition comprising (i) dupilumab at a concentration greater than about 100 mg/ml±10 mg/mL; (ii) a buffer selected from the group consisting of adipic acid, no separate buffer, histidine, or succinic acid; (iii) a component selected from the group consisting of a sugar, and a non-charged amino acid, and mixtures thereof; (iv) a surfactant and (v) a component selected from the group consisting of arginine and lysine.

2. The composition of claim 1, wherein the dupilumab concentration is about 150 mg/mL to about 175 mg/mL.

3. The composition of claim 1, wherein the dupilumab concentration is about 150 mg/mL.

4. The composition of claim 1, wherein the dupilumab concentration is about 175 mg/mL.

5. The composition of claim 5, wherein the composition has a pH of about 5.9.

6. The composition of claim 1, wherein the buffer is adipic acid.

7. The composition of claim 1, comprising no separate buffer.

8. The composition of claim 1, wherein the buffer is histidine.

9. The composition of claim 1, wherein the buffer is succinic acid.

10. The composition of claim 1, wherein the sugar is sorbitol.

11. The composition of claim 1, wherein the sugar is sucrose.

12. The composition of claim 1, wherein the amino acid is methionine.

13. The composition of claim 1, wherein the amino acid is proline.

14. The composition of claim 1, comprising arginine.

15. The composition of claim 1, comprising lysine.

16. The composition of claim 1, wherein the surfactant is polysorbate 80.

17. The composition of claim 1, wherein the surfactant is PLX188.

18. The composition of claim 1, comprising (i) dupilumab at a concentration of 150 or 175 mg/ml; (ii) 10 mM histidine; (iii) 150 mM sucrose; (iv) 2 mg/ml polysorbate 80; and (v) 50 mM arginine.

19. The composition of claim 1, comprising (i) dupilumab at a concentration of 150 or 175 mg/ml; (ii) 10 mM histidine; (iii) 150 mM sucrose; (iv) 2 mg/ml polysorbate 80; (v) 50 mM arginine and (vi) 10 mM methionine.

20. The composition of claim 1, comprising (i) dupilumab at a concentration of 150 or 175 mg/ml; (ii) 10 mM histidine; (iii) 150 mM sucrose; (iv) 2 mg/ml polysorbate 80; and (v) 75 mM lysine.

21. The composition of claim 1, comprising (i) dupilumab at a concentration of 150 or 175 mg/ml; (ii) 10 mM histidine; (iii) 150 mM sorbitol; (iv) 2 mg/ml polysorbate 80; and (v) 50 mM arginine.

22. The composition of claim 1, comprising (i) dupilumab at a concentration of 150 or 175 mg/mL; (ii) 10 mM histidine; (iii) 50 mM sucrose; (iv) 2 mg/ml polysorbate 80; and (v) 150 mM lysine.

23. The composition of claim 1, comprising (i) dupilumab at a concentration of 150 or 175 mg/ml; (ii) 10 mM histidine; (iii) 50 mM NaCl; (iv) 2 mg/ml polysorbate 80; and (v) 150 mM proline.

24. The composition of claim 1, comprising (i) dupilumab at a concentration of 150 or 175 mg/ml; (ii) 10 mM histidine; (iii) 2 mg/ml polysorbate 80; and (iv) 150 mM proline.

25. The composition of claim 1, comprising (i) dupilumab at a concentration of 150 or 175 mg/ml; (ii) 20 mM succinic acid and arginine; (iii) 2 mg/ml polysorbate 80; and (iv) 150 mM sucrose.

26. The composition of claim 1, comprising (i) dupilumab at a concentration of 150 or 175 mg/ml; (ii) 10 mM succinic acid; (iii) 2 mg/ml polysorbate 80; (iv) 75 mM lysine and (v) 150 mM proline.

27. The composition of claim 1, comprising (i) dupilumab at a concentration of 150 or 175 mg/ml; (ii) 10 mM adipic acid; (iii) 150 mM sucrose; (iv) 2 mg/ml polysorbate 80; and (v) 50 mM arginine.

28. The composition of claim 1, comprising (i) dupilumab at a concentration of 150 or 175 mg/ml; (ii) 10 mM succinic acid; (iii) 150 mM proline; (iv) 2 mg/ml polysorbate 80; and (v) 50 mM arginine.

29. The composition of claim 1, comprising (i) dupilumab at a concentration of 150 or 175 mg/ml; (ii) 170 mM sucrose; (iv) 2 mg/ml polysorbate 80; and (v) 50 mM arginine.

30. The composition of claim 1, comprising (i) dupilumab at a concentration of 150 or 175 mg/ml; (ii) 10 mM histidine; (iii) 2 mg/ml polysorbate 80; and (iv) 150 mM arginine.

31. The composition of claim 1, comprising (i) dupilumab at a concentration of 150 or 175 mg/ml; (ii) 170 mM sucrose; (iv) 0.5 mg/ml polysorbate 80; and (v) 50 mM arginine.

32. The composition of claim 1, comprising (i) dupilumab at a concentration of 150 or 175 mg/mL; (ii) 170 mM sucrose; (iv) 0.5 mg/ml PLX188; and (v) 50 mM arginine.

33. The composition of claim 1, comprising (i) dupilumab at a concentration of 150 or 175 mg/ml; (ii) 10 mM histidine; (iii) 2 mg/ml polysorbate 80; and (iv) 150 mM arginine.

34. A method for stabilizing an aqueous high protein concentration composition, wherein the protein has a determined isoelectric point and the composition has a target pH, comprising (i) formulating the composition with a low ionic strength buffer or bufferless system if a difference between the target pH and the IEP is about 2 or greater, and (ii) formulating the composition with a high ionic strength buffer if a difference between the target pH and the IEP, is less than about 2.

35. A method for selecting a buffer system for an aqueous high protein concentration composition, wherein the protein has a determined isoelectric point and the composition has a target pH, comprising determining a difference between the target pH and the IEP, wherein a low ionic strength buffer or bufferless system is selected if the difference between the target pH and the IEP is about 2 or greater, and wherein a high ionic strength buffer is selected if the difference between the target pH and the IEP, is less than about 2.


SEQUENCE LISTING (2 sequences)

SEQ ID NO: 1 - protein, 451 aa, Homo sapiens
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 2 - protein, 219 aa, Homo sapiens
DIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC